(19) United States
(12) Patent Application Publication
(10) Pub. No.: US 2025/0191674 A1
(43) Pub. Date: Jun. 12, 2025

(54) PROTEIN DESIGN WITH SEGMENT PRESERVATION

(71) Applicant: Genentech, Inc., South San Francisco, CA (US)

(72) Inventors: Vladimir GLIGORIJEVIC, New Haven, CT (US); Simon Paul KELOW, Washington, DC (US); Jae Hyeon LEE, Boston, MA (US); Ji Won PARK, Stanford, CA (US); Stephen Robert RA, New York, NY (US); Andrew Martin WATKINS, San Francisco, CA (US); Daniel BERENBERG, New York, NY (US); Richard A. BONNEAU, New York, NY (US); Kyunghyun CHO, New York, NY (US)

(21) Appl. No.: 18/842,226

(22) PCT Filed: Feb. 28, 2023
(86) PCT No.: PCT/US2023/014147

§ 371 Date: Aug. 28, 2024

Related U.S. Application Data
(60) Provisional application No. 63/315,046, filed on Feb. 28, 2022.

(51) Int. Cl. G16B 15/30; G16B 15/20; G16B 40/20

(52) U.S. Cl. CPC G16B 15/30; G16B 15/20; G16B 40/20

(57) ABSTRACT

A method for segment preserving protein design includes determining, within a protein structure having a first sequence of residues, one or more fixed segments and adjustable segments. The protein structure may be identified as having a desired property. A protein design computational model may be used to generate a second sequence of residues comprising at least one of a corruption and a length change to the first adjustable segment. The protein design computational model may be further used to generate a modified protein structure having the second sequence of residues. The second sequence of residues forming the modified protein structure includes the fixed segments present in the first sequence of residues. Structural and/or functional analysis may be performed to determine whether the modified protein structure also exhibits the same desired property as the protein structure. Related systems and computer program products are also provided.

### CROSS REFERENCE TO RELATED APPLICATION

This application claims priority to U.S. Provisional Application No. 63/315,046, entitled “PROTEIN DESIGN WITH SEGMENT PRESERVATION” and filed on Feb. 28, 2022, the disclosure of which is incorporated herein by reference in its entirety.

### TECHNICAL FIELD

The subject matter described herein relates generally to protein design and more specifically to techniques for designing protein sequences in which one or more segments are preserved.

### INTRODUCTION

Proteins are responsible for many essential cellular functions including, for example, enzymatic reactions, transport of molecules, regulation and execution of a number of biological pathways, cell growth, proliferation, nutrient uptake, morphology, motility, intercellular communication, and/or the like. A protein structure may include one or more polypeptides, which are chains of amino acid residues linked together by peptide bonds. The sequence of amino acid residues in the polypeptide chains forming the protein structure determines the protein's three-dimensional structure (e.g., the protein's tertiary structure). Moreover, the sequence of amino acids in the polypeptide chains forming the protein determines the protein's underlying functions. As such, the primary objective of de novo protein design includes constructing one or more sequences of amino acid residues that exhibit certain traits. For example, in the case of large molecule drug discovery, de novo protein design will often seek to identify sequences of amino acid residues (e.g., antibodies and/or the like) capable of binding to an antigen such as a viral antigen, a tumor antigen, and/or the like.

### SUMMARY

Systems, methods, and articles of manufacture, including computer program products, are provided for segment preserving protein design. In some example embodiments, there is provided a system that includes at least one processor and at least one memory. The at least one memory may include program code that provides operations when executed by the at least one processor. The operations may include: determining, within a protein structure having a first sequence of residues, a first fixed segment and a first adjustable segment; identifying a desired property associated with the protein structure; generating, using a protein design computational model, a second sequence of residues comprising at least one of a corruption and a length change to the first adjustable segment; and generating, using the protein design computational model, a modified protein structure having the second sequence of residues.

In some variations, one or more features disclosed herein including the following features can optionally be included in any feasible combination. The protein design computational model may include a machine learning model trained to generate the second sequence of residues.

In some variations, the machine learning model may generate the second sequence of residues by at least sampling a data distribution learned through training.

In some variations, the sampling of the data distribution may include generating a corrupted sequence by modifying the first adjustable segment, encoding the corrupted sequence to generate an encoding having a length corresponding to a quantity of residues present in the encoding, generating an intermediate sequence by altering the length of the encoding of the corrupted sequence while maintaining a length of the first fixed segment, and generating, based at least on a decoding of the intermediate sequence, the second sequence of residues.

In some variations, the corrupted sequence may be generated without modifying the first fixed segment included in the first sequence of residues.

In some variations, the second sequence of residues may include the first fixed segment.

In some variations, the decoding of the intermediate sequence may be generated based at least on an index map identifying the first fixed segment within the intermediate sequence

In some variations, the decoding of the intermediate sequence may include determining, for each position within the intermediate sequence, a probability distribution across a vocabulary of possible amino acid residues.

In some variations, the probability distribution may be determined by applying one or more of autoregressive modeling, non-autoregressive modeling, and condition random fields.

In some variations, the operations may further include: determining, within the protein structure having the first sequence of residues, a second fixed segment; and sampling the data distribution to generate the second sequence of residues to include the first fixed segment and the second fixed segment.

In some variations, the sampling of the data distribution may include generating the corrupted sequence by modifying the first adjustable segment, where the corrupted sequence includes the modified first adjustable segment, the first fixed segment, and the second fixed segment; generating the intermediate sequence by altering the length of the encoding of the corrupted sequence while maintaining the length of the first fixed segment or the second fixed segment; generating an index map to identify the first fixed segment and the second fixed segment within the intermediate sequence; and generating the second sequence of residues to include the first fixed segment and the second fixed segment by decoding the intermediate sequence based on the index map.

In some variations, a difference between a first length of the first sequence of residues and a second length of the second sequence of residues may be distributed amongst the first adjustable segment and a second adjustable segment by at least changing a first length of the first adjustable segment and/or changing a second length of the second adjustable segment.

In some variations, the difference between the first length of the first sequence of residues and the second length of the second sequence of residues may be determined based on a probability distribution of possible length differences between the first sequence of residues and the second sequence of residues.

In some variations, the difference between the first length of the first sequence of residues and the second length of the second sequence of residues may be distributed proportionally to the first length of the first adjustable segment and the second length of the second adjustable segment.

In some variations, the difference between the first length of the first sequence of residues and the second length of the second sequence of residues may be distributed randomly amongst the first adjustable segment and the second adjustable segment.

In some variations, the difference between the first length of the first sequence of residues and the second length of the second sequence of residues may be distributed to the first adjustable segment but not the second adjustable segment such that the second length of the second adjustable second segment is preserved.

In some variations, the difference between the first length of the first sequence of residues and the second length of the second sequence of residues may be distributed by applying no more than a maximum length change and/or no less than a minimum length change to at least one of the first length of the first adjustable segment and the second length of the second adjustable segment.

In some variations, the first sequence of residues may include an antibody. The first segment may include a complementarity determining region (CDR) of the antibody or a non-complementarity determining region of the antibody.

In some variations, an input of the protein design computational model may include one or more identifiers to enable a differentiation between a first portion of the first sequence corresponding to a heavy chain of the antibody and a second portion of the first sequence corresponding to a light chain of the antibody.

In some variations, the input of the protein design computational model may further include the one or more identifiers to enable a differentiation between the first portion of the first sequence corresponding to the heavy chain of the antibody, the second portion of the first sequence corresponding to the light chain of the antibody, and a third portion of the first sequence corresponding to an antigen having a known binding affinity towards the antibody.

In some variations, the third portion of the first sequence may include a fixed segment and/or an adjustable segment.

In some variations, the protein design computational model may generate the second sequence of residues based on the one or more identifiers such that the first fixed segment included in the second sequence of residues is present in an identical chain as the first sequence of residues.

In some variations, the one or more identifiers may include a token between the first portion of the first sequence corresponding to the heavy chain of the antibody and a second portion of the first sequence corresponding to the light chain of the antibody.

In some variations, the one or more identifiers may include a first tag identifying each residue in the heavy chain of the antibody and a second tag identifying each residue in the light chain of the antibody.

In some variations, the corruption may include at least one of inserting a residue into the first adjustable segment, deleting a residue from the first adjustable segment, and modifying a residue present in the first adjustable segment.

In some variations, the data distribution may correspond to a reduced dimension representation of data corresponding to a plurality of known protein sequences. At least a portion of the plurality of sequence of residues may be associated with one or more known functions.

In some variations, the protein design computational model may include an autoencoder.

In some variations, the protein design computational model may include a denoising autoencoder (DAE).

In some variations, the first fixed segment may be determined based at least on the first fixed segment being associated with the desired property.

In some variations, the operations may further include: performing one or more of a structural analysis and a functional analysis to determine that the second sequence of residues exhibits the desired property.

In some variations, the operations may further include: generating a fixed-length representation of the first sequence of residues including the first fixed segment and the first adjustable segment; and applying the protein design computational model to generate the second sequence of residues by at least applying the at least one of the corruption and the length change to the first adjustable segment included in the fixed-length representation of the first sequence of residues.

In some variations, the fixed-length representation of the first sequence of residues may be generated by at least determining, based at least on a multi-sequence alignment including a plurality of known protein sequences, a global index having a plurality of integer positions, and assigning, based at least on the global index aligned to the first sequence of residues, a corresponding integer position from the plurality of integer positions to the each residue included in the first sequence of residues.

In some variations, the fixed-length representation of the input sequence may include a gap character at each integer position where the first sequence of residues fails to include a corresponding residue at the integer position.

In another aspect, there is provided a method for segment-preserving protein design. The method may include: determining, within a protein structure having a first sequence of residues, a first fixed segment and a first adjustable segment; identifying a desired property associated with the protein structure; generating, using a protein design computational model, a second sequence of residues comprising at least one of a corruption and a length change to the first adjustable segment; and generating, using the protein design computational model, a modified protein structure having the second sequence of residues.

In some variations, one or more features disclosed herein including the following features can optionally be included in any feasible combination. The protein design computational model may include a machine learning model trained to generate the second sequence of residues.

In some variations, the machine learning model may generate the second sequence of residues by at least sampling a data distribution learned through training.

In some variations, the sampling of the data distribution may include generating a corrupted sequence by modifying the first adjustable segment, encoding the corrupted sequence to generate an encoding having a length corresponding to a quantity of residues present in the encoding, generating an intermediate sequence by altering the length of the encoding of the corrupted sequence while maintaining a length of the first fixed segment, and generating, based at least on a decoding of the intermediate sequence, the second sequence of residues.

In some variations, the corrupted sequence may be generated without modifying the first fixed segment included in the first sequence of residues.

In some variations, the second sequence of residues may include the first fixed segment.

In some variations, the decoding of the intermediate sequence may be generated based at least on an index map identifying the first fixed segment within the intermediate sequence

In some variations, the decoding of the intermediate sequence may include determining, for each position within the intermediate sequence, a probability distribution across a vocabulary of possible amino acid residues.

In some variations, the probability distribution may be determined by applying one or more of autoregressive modeling, non-autoregressive modeling, and condition random fields.

In some variations, the method may further include: determining, within the protein structure having the first sequence of residues, a second fixed segment; and sampling the data distribution to generate the second sequence of residues to include the first fixed segment and the second fixed segment.

In some variations, the sampling of the data distribution may include generating the corrupted sequence by modifying the first adjustable segment, where the corrupted sequence includes the modified first adjustable segment, the first fixed segment, and the second fixed segment; generating the intermediate sequence by altering the length of the encoding of the corrupted sequence while maintaining the length of the first fixed segment or the second fixed segment; generating an index map to identify the first fixed segment and the second fixed segment within the intermediate sequence; and generating the second sequence of residues to include the first fixed segment and the second fixed segment by decoding the intermediate sequence based on the index map.

In some variations, a difference between a first length of the first sequence of residues and a second length of the second sequence of residues may be distributed amongst the first adjustable segment and a second adjustable segment by at least changing a first length of the first adjustable segment and/or changing a second length of the second adjustable segment.

In some variations, the difference between the first length of the first sequence of residues and the second length of the second sequence of residues may be determined based on a probability distribution of possible length differences between the first sequence of residues and the second sequence of residues.

In some variations, the difference between the first length of the first sequence of residues and the second length of the second sequence of residues may be distributed proportionally to the first length of the first adjustable segment and the second length of the second adjustable segment.

In some variations, the difference between the first length of the first sequence of residues and the second length of the second sequence of residues may be distributed randomly amongst the first adjustable segment and the second adjustable segment.

In some variations, the difference between the first length of the first sequence of residues and the second length of the second sequence of residues may be distributed to the first adjustable segment but not the second adjustable segment such that the second length of the second adjustable second segment is preserved.

In some variations, the difference between the first length of the first sequence of residues and the second length of the second sequence of residues may be distributed by applying no more than a maximum length change and/or no less than a minimum length change to at least one of the first length of the first adjustable segment and the second length of the second adjustable segment.

In some variations, the first sequence of residues may include an antibody. The first segment may include a complementarity determining region (CDR) of the antibody or a non-complementarity determining region of the antibody.

In some variations, an input of the protein design computational model may include one or more identifiers to enable a differentiation between a first portion of the first sequence corresponding to a heavy chain of the antibody and a second portion of the first sequence corresponding to a light chain of the antibody.

In some variations, the input of the protein design computational model may further include the one or more identifiers to enable a differentiation between the first portion of the first sequence corresponding to the heavy chain of the antibody, the second portion of the first sequence corresponding to the light chain of the antibody, and a third portion of the first sequence corresponding to an antigen having a known binding affinity towards the antibody.

In some variations, the third portion of the first sequence may include a fixed segment and/or an adjustable segment.

In some variations, the protein design computational model may generate the second sequence of residues based on the one or more identifiers such that the first fixed segment included in the second sequence of residues is present in an identical chain as the first sequence of residues.

In some variations, the one or more identifiers may include a token between the first portion of the first sequence corresponding to the heavy chain of the antibody and a second portion of the first sequence corresponding to the light chain of the antibody.

In some variations, the one or more identifiers may include a first tag identifying each residue in the heavy chain of the antibody and a second tag identifying each residue in the light chain of the antibody.

In some variations, the corruption may include at least one of inserting a residue into the first adjustable segment, deleting a residue from the first adjustable segment, and modifying a residue present in the first adjustable segment.

In some variations, the data distribution may correspond to a reduced dimension representation of data corresponding to a plurality of known protein sequences. At least a portion of the plurality of sequence of residues may be associated with one or more known functions.

In some variations, the protein design computational model may include an autoencoder.

In some variations, the protein design computational model may include a denoising autoencoder (DAE).

In some variations, the first fixed segment may be determined based at least on the first fixed segment being associated with the desired property.

In some variations, the method may further include: performing one or more of a structural analysis and a functional analysis to determine that the second sequence of residues exhibits the desired property.

In some variations, the operations may further include: generating a fixed-length representation of the first sequence of residues including the first fixed segment and the first adjustable segment; and applying the protein design computational model to generate the second sequence of residues by at least applying the at least one of the corruption and the length change to the first adjustable segment included in the fixed-length representation of the first sequence of residues.

In some variations, determining, based at least on a multi-sequence alignment including a plurality of known protein sequences, a global index having a plurality of integer positions, and assigning, based at least on the global index aligned to the first sequence of residues, a corresponding integer position from the plurality of integer positions to the each residue included in the first sequence of residues.

In some variations, the fixed-length representation of the input sequence may include a gap character at each integer position where the first protein sequence fails to include a corresponding residue at the integer position.

In another aspect, there is provided a computer program product including a non-transitory computer readable medium storing instructions. The instructions may cause operations may executed by at least one data processor. The operations may include: determining, within a protein structure having a first sequence of residues, a first fixed segment and a first adjustable segment; identifying a desired property associated with the protein structure; generating, using a protein design computational model, a second sequence of residues comprising at least one of a corruption and a length change to the first adjustable segment; and generating, using the protein design computational model, a modified protein structure having the second sequence of residues.

In another aspect, there is provided a system that includes at least one data processor and at least one memory. The at least one memory may store instructions that result in operations when executed by the at least one data processor. The operations may include: identifying, within a first antibody having a first sequence of residues, a first fixed segment associated with a first desired property of the first antibody, generating a second sequence of residues to include the first fixed segment and a first adjustable segment; applying a protein design computational model to generate a third sequence of residues to include the first fixed segment and at least one of a corruption and a length change to the first adjustable segment; applying a property prediction model to determine a second desired property exhibited by the third sequence of residues; and generating, based at least on the second desired property of the third sequence of residues satisfying one or more thresholds, a second antibody having the third sequence of residues.

In another aspect, there is provided a method that includes: identifying, within a first antibody having a first sequence of residues, a first fixed segment associated with a first desired property of the first antibody; generating a second sequence of residues to include the first fixed segment and a first adjustable segment; applying a protein design computational model to generate a third sequence of residues to include the first fixed segment and at least one of a corruption and a length change to the first adjustable segment; applying a property prediction model to determine a second desired property exhibited by the third sequence of residues; and generating, based at least on the second desired property of the third sequence of residues satisfying one or more thresholds, a second antibody having the third sequence of residues.

In another aspect, there is provided a computer program product including a non-transitory computer readable medium storing instructions. The instructions may cause operations may executed by at least one data processor. The operations may include: identifying, within a first antibody having a first sequence of residues, a first fixed segment associated with a first desired property of the first antibody; generating a second sequence of residues to include the first fixed segment and a first adjustable segment; applying a protein design computational model to generate a third sequence of residues to include the first fixed segment and at least one of a corruption and a length change to the first adjustable segment; applying a property prediction model to determine a second desired property exhibited by the third sequence of residues; and generating, based at least on the second desired property of the third sequence of residues satisfying one or more thresholds, a second antibody having the third sequence of residues.

In some variations of the methods, systems, non-transitory computer readable media, and computer-implemented methods, one or more features disclosed herein including the following features can optionally be included in any feasible combination.

In some variations, the property prediction model may be applied to determine the first desired property exhibited by the third sequence of residues. The second antibody having the third sequence of residues may be generated based at least on the first desired property of the third sequence of residues satisfying the one or more thresholds.

In some variations, the first desired property may be a binding affinity towards a target molecule and the second desired property may be one or more of expression, non-specificity, stability, non-immunogenicity, human-ness, and self-association.

In some variations, the first antibody may be a non-human antibody.

In some variations, the first fixed segment may include a complementarity determining region (CDR) of the first antibody.

In some variations, the first fixed segment may include one or more Vernier zone residues in the first antibody.

In some variations, the first adjustable segment may include a randomly generated sequence of amino acid residues.

In some variations, the first adjustable segment may include a framework region of a human antibody.

In some variations, the first adjustable segment may include a framework region of a human antibody without one or more Vernier zone residues.

In some variations, a second fixed segment associated with the first desired property of the first antibody may be identified within the first antibody having the first sequence of residues. The second sequence of residues may be generated to include the second fixed segment. The protein design computational model may be applied to generate the third sequence of residues to include the first fixed segment and the second fixed segment.

In some variations, the second sequence of residues may be generated to include a second adjustable segment. The protein design computational model may be applied to generate the third sequence of residues to further include the at least one of the corruption and the length change to the first adjustable segment and/or the second adjustable segment.

In some variations, the length change may be distributed amongst the first adjustable segment and the second adjustable segment.

In another aspect, there is provided a system that includes at least one data processor and at least one memory. The at least one memory may store instructions that result in operations when executed by the at least one data processor. The operations may include: identifying, within a first protein structure having a first sequence of residues, an adjustable segment associated with one or more undesired properties of the first protein structure; generating a second sequence of residues to include the adjustable segment and a fixed segment; applying a protein design computational model to generate a third sequence of residues to include the fixed segment and at least one of a corruption and a length change to the adjustable segment; applying a property prediction model to determine the one or more undesired properties exhibited by the third sequence of residues; and generating, based at least on the one or more undesired properties of the third sequence of residues satisfying one or more thresholds, a second protein structure having the third sequence of residues.

In another aspect, there is provided a method that includes: identifying, within a first protein structure having a first sequence of residues, an adjustable segment associated with one or more undesired properties of the first protein structure; generating a second sequence of residues to include the adjustable segment and a fixed segment; applying a protein design computational model to generate a third sequence of residues to include the fixed segment and at least one of a corruption and a length change to the adjustable segment; applying a property prediction model to determine the one or more undesired properties exhibited by the third sequence of residues; and generating, based at least on the one or more undesired properties of the third sequence of residues satisfying one or more thresholds, a second protein structure having the third sequence of residues.

In another aspect, there is provided a computer program product including a non-transitory computer readable medium storing instructions. The instructions may cause operations may executed by at least one data processor. The operations may include: identifying, within a first protein structure having a first sequence of residues, an adjustable segment associated with one or more undesired properties of the first protein structure; generating a second sequence of residues to include the adjustable segment and a fixed segment; applying a protein design computational model to generate a third sequence of residues to include the fixed segment and at least one of a corruption and a length change to the adjustable segment; applying a property prediction model to determine the one or more undesired properties exhibited by the third sequence of residues; and generating, based at least on the one or more undesired properties of the third sequence of residues satisfying one or more thresholds, a second protein structure having the third sequence of residues.

In some variations of the methods, systems, non-transitory computer readable media, and computer-implemented methods, one or more features disclosed herein including the following features can optionally be included in any feasible combination.

In some variations, the adjustable segment may include an amino acid residue or a pattern of amino acid residues associated with the one or more undesired properties.

In some variations, the protein design computation model may be applied to generate the third sequence of residues by at least replacing and/or removing the amino acid residue or the pattern of amino acid residues associated with the one or more undesired properties.

In some variations, the one or more undesired properties may include a propensity for oxidation, chemical modification, and/or chemical isomerization.

In some variations, the one or more undesired properties may include immunogenicity.

In some variations, the fixed segment may be identified for inclusion in the second sequence of residues based at least on the fixed segment being associated with one or more desirable properties.

In some variations, the one or more desirable properties may include a binding affinity towards a target molecule, expression, non-specificity, stability, non-immunogenicity, human-ness, and/or self-association.

In some variations, the fixed segment may include a complementarity determining region (CDR) and/or one or more Vernier zone residues.

In some variations, the property prediction model may be applied to determine one or more desired properties exhibited by the third sequence of residues. The second protein structure having the third sequence of residues may be generated based at least on the one or more desired properties of the third sequence of residues satisfying the one or more thresholds.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to segment preserving protein design, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a protein design system, in accordance with some example embodiments;

FIG. 2A depicts a flowchart illustrating an example of a process for segment-preserving protein design, in accordance with some example embodiments;

FIG. 2B depicts a flowchart illustrating another example of a process for segment-preserving protein design, in accordance with some example embodiments;

FIG. 2C depicts a flowchart illustrating another example of a process for segment-preserving protein design, in accordance with some example embodiments;

FIG. 3A depicts a schematic diagram illustrating examples of protein sequences, in accordance with some example embodiments;

FIG. 3B depicts a schematic diagram illustrating examples of input protein sequences and output protein sequences, in accordance with some example embodiments; and

FIG. 4 depicts a block diagram illustrating an example of a computing system, in accordance with some example embodiments.

When practical, similar reference numbers denote similar structures, features, or elements.

### DETAILED DESCRIPTION

De novo protein design aims to identify protein sequences (e.g., sequences of amino acid residues) that exhibit certain functionalities, such as binding affinity towards another molecule (e.g., a viral antigen, a tumor antigen, and/or the like). Nevertheless, de novo protein design is a challenging and resource intensive task at least because the combinatorial search space of every possible permutation of amino acid residues that can form a protein structure is vast but sparsely populated by sequences of amino acid residues that correspond to actually functional proteins. That is, the vast majority of protein sequences in the combinatorial search space will not exhibit any function at all, let alone a desired property such as a binding affinity towards certain molecules. Moreover, this combinatorial search space becomes even more immense when considering candidate protein sequences having variable lengths (e.g., candidate protein sequences with different quantities of amino acid residues). Thus, a brute force approach that indiscriminately examines every possible sequence of amino acid residues to identify sequences that exhibit a desired property, even when performed in silico, is too computationally expensive to be a feasible solution.

In some example embodiments, instead of exploring a vast combinatorial space that is sparsely populated by functional protein sequences, a protein design engine may generate one or more protein sequences (e.g., sequences of amino acid residues) by sampling a data distribution associated with various known protein sequences, including those that are known to be functional. For example, the protein design engine may include a machine learning model that is trained using known protein sequences including protein sequences known to exhibit certain functions and protein sequences without any known functions. In doing so, the machine learning model may learn a data distribution corresponding to a reduced dimension representation of the sequences of amino acid residues forming the known protein sequences. The data distribution in this case may be topological space (e.g., a manifold) occupied by the known protein sequences that describes the relationships between the known protein sequences. In particular, the high dimensionality of the data associated with the known protein sequences may obscure the relationships between populations of protein sequences having structural similarities. These relationships may include the density of each population of protein sequences exhibiting a similar structure and the magnitude of structural similarities between adjacent populations of protein sequences within the data distribution. The data distribution learned by the machine learning model, which reduces the dimensionality of the data associated with the protein sequences, may therefore enable the identification of one or more populations of protein sequences that exhibit structural similarities.

In some example embodiments, the machine learning model may be trained to learn a manifold occupied by the protein sequences with a high probability of being functional. Moreover, at inference time during which the trained machine learning model is applied, for example, by sampling the data distribution to identify one or more candidate protein sequences, which are then subjected to further functional and/or structural analysis to determine whether each candidate protein sequence exhibits the desired property. Because the data distribution (e.g., the manifold) include protein sequences with a high probability of being functional, the protein design engine is more likely to identify candidate protein sequences that are functional when sampling the data distribution, thus increasing the computational efficiency of generating functional protein sequences in silico.

In some example embodiments, the protein design engine may generate, based on one protein sequence having a desired property, one or more additional protein sequences having a same (or similar) property. For example, the trained machine learning model may be applied by sampling, based on a first protein sequence exhibiting a desired property, the data distribution to generate a second protein sequence also exhibiting the same desired property. The sampling of the data distribution may be performed based on an intermediate sequence having at least one of a corruption (e.g., an insertion, a deletion, and/or a modification of an amino acid residue) or a length change relative to the first protein sequence. In cases where the machine learning model is implemented by an autoencoder (e.g., a denoising autoencoder (DAE) and/or the like), training the machine learning model to learn the data distribution may include training the encoder to generate an encoding of an input protein sequence that can be decoded by the decoder to form an output protein sequence that is minimally different from the input protein sequence. Here, the encoding of the input protein sequence may correspond to a representation of the input protein sequence in the reduced dimension space of the data distribution whereas the subsequent decoding corresponds to a projection back to the higher dimensional space of the original input protein sequence.

At inference time, the sampling of the data distribution may include encoding a first protein sequence exhibiting a desired property before decoding an intermediate sequence having at least one of a corruption (e.g., an insertion, a deletion, and/or a modification of an amino acid residue) or a length change relative to the first protein sequence. The decoding of the intermediate sequence may generate a second protein sequence that is different than the first protein sequence but is still likely to exhibit a same (or similar) function as the first protein sequence. Accordingly, in some cases, the second protein sequence may be subjected to further functional and/or structural analysis to determine the functions associated with the second protein sequence, with the results of the functional and/or structural analysis used as feedback to guide subsequent sampling of the data distribution.

In some cases, the desired properties of a protein sequence may be attributable to one or more segments (e.g., sub-sequences of amino acid residues) present within the protein sequence. For example, the ability of an antibody to bind to certain target molecules (e.g., tumor antigens, viral antigens, and/or the like) may be attributable to the sub-sequences of amino acid residues forming the complementarity determining regions (CDRs) and/or Vernier zone residues of the antibody protein sequence. Alternatively and/or additionally, one or more undesired properties of a protein sequence may also be attributable to one or more segments present within the protein sequence. For instance, tryptophan residues may be prone to oxidization under chemical stress, “NP” motifs may be prone to chemical modification by protease enzymes, and aspartate residues may be prone to chemical isomerization while in formulation. As such, in some cases, the overall functionality of a protein sequence may be optimized by at least designating some segments of the protein sequence for preservation and other segments of the protein sequence for modification. In the case of antibody design, for example, an antibody may be generated to include the complementarity determining regions (CDRs) and/or Vernier zone residues of another antibody exhibiting the desired binding affinity towards one or more target molecules but without the tryptophan residues, “NP” motifs, and aspartate residues associated with the aforementioned chemical liabilities.

Accordingly, in some example embodiments, the protein design engine may leverage a priori biological, chemical, and/or physical knowledge to impose certain constraints on the sampling the data distribution. For example, a priori biological, chemical, and/or physical knowledge may indicate that certain segments of a first protein sequence are associated with a desired property, in which case the protein design engine may be configured to preserve these segments when generating a second protein sequence in order to avoid reducing (or eliminating) the desired property in the second protein sequence. That is, preserving the segments associated with the desired property when generating the second protein structure may increase (or maximize) the likelihood that the second protein sequence also exhibits the same desired property. Alternatively and/or additionally, a priori biological, chemical, and/or physical knowledge may indicate that certain segments of the first protein sequence are associated with a undesired property, in which case the protein design engine may be configured to modify (or remove) these segments when generating the second protein sequence. Modifying (or removing) the segments associated with the undesired property may decrease (or minimize) the likelihood that the second protein sequence exhibits the undesired property.

As used herein, the term “fixed segment” may refer to a sub-sequence of amino acid residues within the first protein sequence that is preserved or kept constant (e.g., in order, composition, and nature), when generating the second protein sequence. Fixed segments may be preserved at least because these segments are associated with one or more desired properties of the first protein sequence. Contrastingly, the first protein sequence may also include one or more “adjustable segments,” which are sub-sequences of amino acid residues in the first protein sequence that may be changed, either in their nature, composition, or order, during the generation of the second protein sequence. In other words, the “adjustable segments” are not necessarily preserved during the generation of the second protein sequence. Moreover, differences between the first protein sequence and the second protein sequence, such as the insertion, deletion, and/or modification of one or more amino acid residues and the concomitant length changes, may be confined to these “adjustable segments.” Contrastingly, the same “fixed segments” present in the first protein sequence may also be present in the second protein sequence.

As noted, an adjustable segment is not necessarily preserved during the generation of the second protein sequence. Moreover, it should be appreciated that it may be the case that an adjustable segment is also not necessarily modified during the generation of the second protein sequence. However, in some cases, an adjustable segment (or a portion of an adjustable segment such as one or more amino acid residues contained therein) may be associated with certain undesired properties. As such, in some cases, at least a portion of an adjustable segment may be designated for modification in order to reduce, minimize, and/or eliminate the corresponding undesired properties. For example, in addition to or instead of preserving certain fixed segments within the first protein sequence in order to preserve certain desired properties of the first protein sequence when generating the second protein sequence, one or more adjustable segments (or portions of the one or more adjustable segments) in the first protein sequence may be designated for modification in order to further preserve the desired properties and/or reduce (or eliminate) certain undesired properties.

Certain undesirable properties may be attributable to the presence of certain amino acid residues or patterns of amino acid residues (formed by amino acid residues occupying adjacent as well as non-adjacent positions) within the first protein sequence. For example, tryptophan residues may be prone to oxidization under chemical stress, “NP” motifs may be prone to chemical modification such as hydrolysis, and aspartate residues may be prone to chemical isomerization while in formulation. Accordingly, in some cases, the generating of the second protein sequence may include preserving the fixed segments associated with the desired properties as well as modifying the amino acid residues (or combination of amino acid residues) associated with the undesired properties.

As noted, in some example embodiments, the protein design engine may generate, based on a first protein sequence having at least one fixed segment, a second protein sequence in which the at least one fixed segment is preserved. In particular, the protein design engine may generate the second protein structure by sampling the data distribution based on an intermediate sequence in which the corruption (e.g., an insertion, a deletion, and/or a modification of an amino acid residue) and/or the length change are applied to one or more adjustable segments within the first protein sequence.

In instances where multiple adjustable segments are present within the first protein sequence, any length difference between the first protein sequence and the second protein sequence may be distributed amongst the adjustable segments. For example, the difference between a first length of the first protein sequence and a second length of the second protein structure may be distributed proportionally amongst the adjustable segments within the first protein sequence based on the respective lengths of the adjustable segments. Alternatively, the difference between the first length of the first protein sequence and the second length of the second protein structure may be distributed randomly amongst the adjustable segments. In some cases, maintaining the desired property within the second protein sequence may require maintaining a certain number of amino acid residues between two successive fixed segments, for example, by preserving the length of an adjustable segment between the two successive fixed segments, applying no more and/or no less than a threshold length change to the adjustable segment, and/or the like. The length of an adjustable segment may be preserved during the generation of the second protein sequence by distributing the difference between the first length of the first protein sequence and the second length of the second protein structure to some but not all of the adjustable segments within the first protein sequence. Alternatively and/or additionally, the difference between the first length of the first protein sequence and the second length of the second protein structure may be distributed in accordance with a maximum length change and/or a minimum length change to one or more of the adjustable sequences.

In some example embodiments, the protein design computational model may ingest a fixed-length representation of the first protein sequence in order to accommodate length changes as a part of the generative process. In some cases, the fixed-length representation of the first protein sequence may be determined based on an alignment of multiple known protein sequences (e.g., a multi-sequence alignment) such as protein sequences from a same protein family (e.g., antibody, antigen-binding fragment (Fab), T-cell receptor (TCR), and/or the like). For example, a global index may be determined based on multi-sequence alignment in which the global index includes a integer position for each position observed in at least one of the protein sequences. In some instances, the global index may a plurality of positions, each of which corresponding to a structural role observed in at least one of the protein sequences. For instance, in some cases, the first protein sequence may be rendered in a fixed length representation by applying a structural role based numbering scheme in which each amino acid residue in the first protein sequence is assigned an integer position in the fixed length sequence (e.g., selected from a range of integers such as [1, 149]) based on the residue's structural role. A gap at any position in the fixed-length sequence where the first protein sequence lacks an amino acid residue having the corresponding structural role may be represented by a gap character or, in some cases, a “ghost residue.” As such, each position in the fixed-length representation of the first protein sequence may be occupied by one of twenty possible amino acid residues or a gap character (e.g., a ghost residue and/or the like). A length change to an adjustable segment of the first protein sequence include inserting an amino acid residue by at least replacing a gap character (e.g., ghost residue and/or the like) in the fixed-length representation of the first protein sequence with the amino acid residue and deleting an amino acid residue by at least replacing the amino acid residue in the fixed-length representation of the first protein sequence with a gap character (e.g., a ghost residue and/or the like).

In some example embodiments, the protein design engine may be configured to generate, based on a first antibody exhibiting a desired property (e.g., expression, binding affinity towards a target molecule, non-specificity, stability, non-immunogenicity, human-ness, self-association, and/or the like), a second antibody exhibiting the same (or similar) desired property. Since the desired property of the first antibody may be attributable to one or more complementarity determining regions (CDRs) of the first antibody, the protein design engine may preserve one or more fixed segments corresponding to the complementarity determining regions (CDRs) of the first antibody when generating the second antibody. Alternatively, the one or more fixed segments may also correspond to the framework regions of the first antibody, which are the non-complementarity determining regions (CDRs) of the first antibody. For example, the protein design engine may generate the second antibody by sampling the data distribution based on an intermediate sequence generated by applying a corruption and/or a length change to one or more adjustable segments of the first antibody. The sampling of the data distribution may generate the second antibody to include the fixed segments of the first antibody (e.g., sub-sequences of amino acid residues corresponding to the complementarity determining regions (CDRs) of the first antibody or the framework regions of the first antibody).

Each antibody may be a protein sequence in which a first portion of the protein sequence corresponds to a heavy chain of the antibody and a second portion of the protein sequence corresponds to a light chain of the antibody. In some cases, in addition to preserving certain fixed segments within the first antibody, preserving the desired property of the first antibody when generating the second antibody may require keeping the fixed segments within an identical chain in the second antibody. Accordingly, in some example embodiments, the input provided to the machine learning model to sample the data distribution may include one or more identifiers (e.g., separator tokens, tags, and/or the like) configured to enable a differentiation between a first portion of the first antibody corresponding to the heavy chain of the first antibody and a second portion of the first antibody corresponding to the light chain of the first antibody. The generating of the second antibody, which includes encoding and decoding an intermediate sequence corresponding to the first antibody, may be performed based on the one or more identifiers such that a fixed segment present on the heavy chain (or light chain) of the first antibody remains on the identical chain in the second antibody.

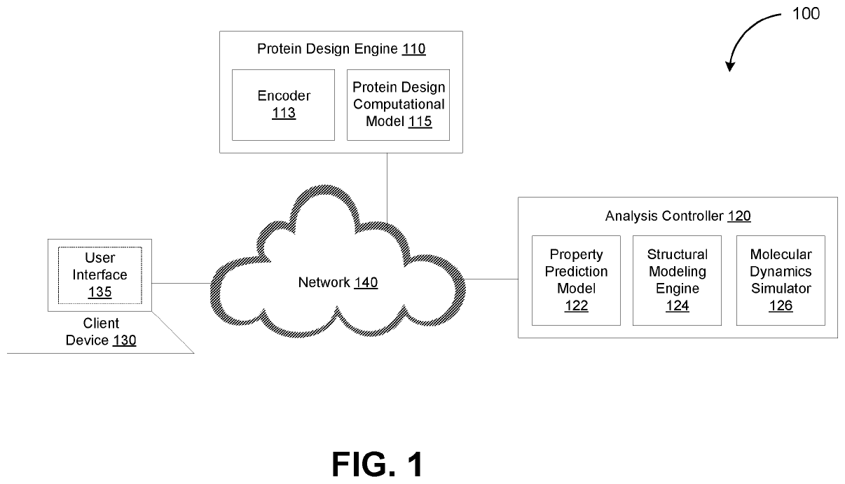

FIG. 1 depicts a system diagram illustrating an example of a protein design system 100, in accordance with some example embodiments. Referring to FIG. 1, the protein design system 100 may include a protein design engine 110, an analysis controller 120, and a client device 130. As shown in FIG. 1, the protein design engine 110, the analysis controller 120, and the client device 130 may be communicatively coupled via a network 140. The client device 130 may be a processor-based device including, for example, a workstation, a desktop computer, a laptop computer, a smartphone, a tablet computer, a wearable apparatus, and/or the like. The network 140 may be a wired network and/or a wireless network including, for example, a local area network (LAN), a virtual local area network (VLAN), a wide area network (WAN), a public land mobile network (PLMN), the Internet, and/or the like.

In some example embodiments, the protein design engine 110 may generate, based on a first protein sequence having a desired property, a second protein sequence having a same (or similar) function. For example, as shown in FIG. 1, the protein design engine 110 may include an encoder 113 and a protein design computational model 115. In some example embodiments, the encoder 113 may generate, based at least on the first protein sequence, a representation of the first protein sequence. In some cases, this representation may be a fixed-length representation of the first protein sequence that have a same length (e.g., quantity of positions) regardless of the quantity of amino acid residues forming the first protein sequence.

In some cases, the fixed-length representation of the first protein sequence may be determined based on an alignment of multiple protein sequences (e.g., a multi-sequence alignment) such as protein sequences from a same protein family (e.g., antibody, antigen-binding fragment (Fab), T-cell receptor (TCR), and/or the like). For example, in some cases, two or more protein sequences may be aligned by applying a sequence alignment technique such as dynamic programming, progressive or hierarchical alignment, iterative alignment, motif finding, Hidden Markov models, and/or the like. A global index having a plurality of integer positions may be determined based on this multi-sequence alignment. Moreover, each integer position within the global index may correspond to a position that is observed in at least one of the protein sequences in the multi-sequence alignment. Accordingly, in some cases, the global index may include an integer position where at least one protein sequence in the multi-sequence alignment includes an amino acid residue at that integer position. It should be appreciated that the global index may include the integer position even in instances where one or more other protein sequences in the multi-sequence alignment does not include an amino acid residue at that integer position.

In some example embodiments, the fixed-length representation of the first protein sequence may be determined by at least aligning the first protein sequence to the global index. It should be appreciated that there may be instances where the first protein sequence does not include an amino acid residue at every integer position within the global index. Accordingly, when generating the fixed-length representation of the first protein sequence based on the global index, the resulting fixed-length representation of the first protein sequence may include one or more where the first protein sequence fails to include an amino acid residue at an integer position present in the global index. These gaps may be represented by one or more corresponding gap characters (e.g., ghost residues and/or the like). As will be explained in more details below, changes to the length of one or more adjustable segments within the first protein sequence may be achieved through the addition and/or removal of gap characters (e.g., ghost residues and/or the like).

In some cases, each integer position within the global index may be associated with a structural role. For example, in some cases, the encoder 113 may apply a structural role based numbering scheme in order to generate the representation of the first protein sequence. In instances where the first protein sequence corresponds to an immunoglobulin protein (or antibody), these structural roles may correspond to the amino acid residue occupying a particular complementarity determining region (CDR) loop or a framework region between a pair of complementarity determining region (CDR) loops. At any position of the representation where the first protein sequence lacks an amino acid residue having the structural role associated with that position, the representation of the first protein sequence may include a gap character (e.g., a ghost residue and/or the like) to represent the corresponding gap.

In some example embodiments, the protein design computational model 115 may be implemented using one or more machine learning models trained to generate the second protein sequence by sampling, based on the first protein sequence (or a fixed length representation of the first protein sequence generated by the encoder 113), a data distribution learned by the one or more machine learning models during training. The one or more machine learning models may be trained based on a variety of known protein sequences, including protein sequences known to exhibit certain functions and protein sequences without any known functions. For instance, in some cases, the protein design computational model 115 may be trained based on known antibodies or subsets of known antibodies, such as antibodies of certain germlines or species (e.g., human antibodies and/or the like). In doing so, the one or more machine learning models may learn a data distribution corresponding to a reduced dimension representation of the sequences of amino acid residues forming the known protein sequences. In particular, the one or more machine learning models may learn the conditional probability between various sub-segments of the known protein sequences including, for example, epistasis mutations in which the effects of mutating a first amino acid residue is dependent on the presence or absence of mutations in one or more other amino acid residues in the same sequence.

When trained based on known antibodies (or known antibodies of specific germlines or species), the one or more machine learning models may learn the conditional probability between types of residues present in the complementarity determining regions (CDRs), the Vernier zones, and the framework regions of various antibodies such that at inference time, the one or more machine learning models may output sequences of residues that are novel yet still consistent with what was observed in the known antibodies. In instances where the one or more machine learning models are trained based on antibodies of a specific germline or species (e.g., human antibodies), the sequences of residues generated by the one or more machine learning models may include amino acid residues (or patterns of amino acid residues) that are mutually compatible for retaining some desired properties (e.g., binding affinity towards a target molecules). In some cases, in addition to preserving certain desired properties, the one or more machine learning models may generate sequence of residues in which the amino acid residues (or patterns of amino acid residues) are mutually compatible for enhancing certain other desired properties (e.g., human-ness, expression, thermostability, and/or the like) and/or reducing various undesired properties (e.g., chemical or drug development liabilities).

In cases where the protein design computational model 115 is implemented using an autoencoder (e.g., a denoising autoencoder (DAE) and/or the like), the protein design computational model 115 may learn the data distribution by learning to generate an encoding of an input protein sequence that can be decoded to form an output protein sequence that is minimally different from the input protein sequence. At inference time, the data distribution associated with the trained protein design computational model 115 may be sampled by encoding a first protein sequence exhibiting a desired property before decoding an intermediate sequence having at least one of a corruption (e.g., an insertion, a deletion, and/or a modification of an amino acid residue) or a length change relative to the first protein sequence. Moreover, the sampling of the data distribution may include decoding the intermediate sequence to generate a second protein sequence that is different than the first protein sequence but is still likely to exhibit a same (or similar) function as the first protein sequence.

Each sampling of the data distribution may correspond to a single sampling iteration generating at least one candidate protein sequence for subsequent structural and/or functional analysis, for example, by the analysis controller 120. The protein design engine 110 may continue to sample the data distribution until one or more conditions are satisfied including, for example, the identification of a threshold quantity of candidate protein sequences, the identification of a threshold quantity of protein sequences exhibiting a desired property, and/or the like. It should be appreciated that the protein design engine 110 may apply a variety of techniques to sample from the data distribution including, for example, a Markov Chain Monte Carlo (MCMC), importance sampling (IS), rejection sampling, Metropolis-Hastings, Gibbs sampling, slice sampling, exact sampling, and/or the like. Moreover, as shown in FIG. 1, the analysis controller 120 may analyze the second protein sequence by applying one or more of a property prediction model 122 (e.g., to evaluate one or more properties of the second protein sequence), structural modeling engine 124 (e.g., to determine a secondary structure and/or a tertiary structure of the second protein sequence), and molecular dynamics simulator 126 (e.g., to determine an energy state and stability of the second protein sequence). At least a portion of the results associated with the sampling, the functional analysis, and/or the structural analysis may be provided for display, for example, in a user interface 135 at the client device 130.

In cases where the desired property of the first protein sequence is attributable to one or more segments (e.g., sub-sequences of amino acid residues) present within the first protein sequence, the protein design engine 110 may generate the second protein sequence to also include the segments associated with the desired property. Doing so may increase (or maximize) the likelihood that the second protein sequence also exhibits the same desired property. As noted, a segment in the first protein sequence may be referred to as a “fixed segment” at least because such segments, or more specifically the sub-sequences of amino acid residues forming each segment, are preserved when generating the second protein sequence. Contrastingly, a segment in the first protein sequence may be referred to as an “adjustable segment” when that segment is not necessarily preserved during the generation of the second protein sequence.

In some example embodiments, the protein design engine 110 may identify the one or more fixed segments and/or adjustable segments in a variety of ways including by leveraging a variety of a priori experimental, biological, chemical, and/or physical knowledge. For example, in some cases, the one or more fixed segments may include a binding interface of an antibody-antigen complex whose structure is determined in vitro and/or in silico (e.g., by the molecular dynamics simulator and/or structure prediction algorithm 126). Alternatively and/or additionally, the one or more fixed segments may include one or more residues identified from an analysis of a protein structure as having structural significance. One such example include residues making hydrogen bonding interactions between the framework region (FR) and complementarity determining region (CDR) of an antibody. At least some residues included in the one or more fixed segments due to their association with certain properties (e.g., residues involved in binding) may be identified and validated experimentally, for example, by surface plasmon resonance (SPR) measurement upon mutation, alanine scanning epitope characterization (e.g., high-throughput mutagenesis), and/or the like. In some cases, at least some residues included in the one or more fixed segments due to their association with certain properties (e.g., residues involved in binding) may be identified through computational means (e.g., computational oracles such as the property prediction model 122).

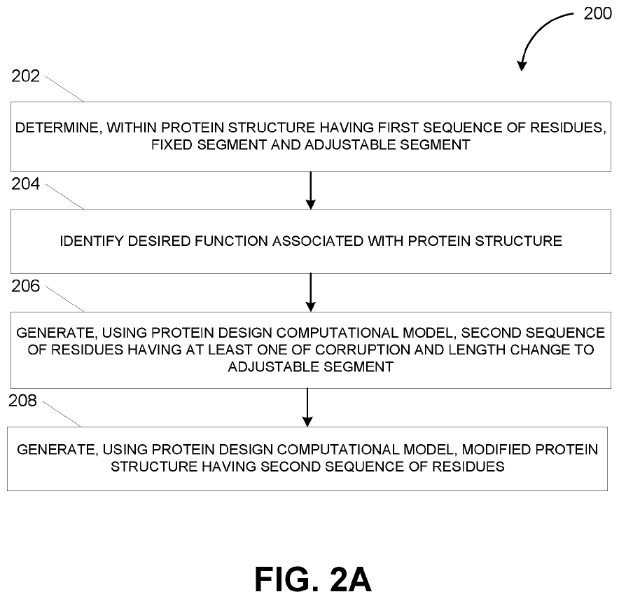

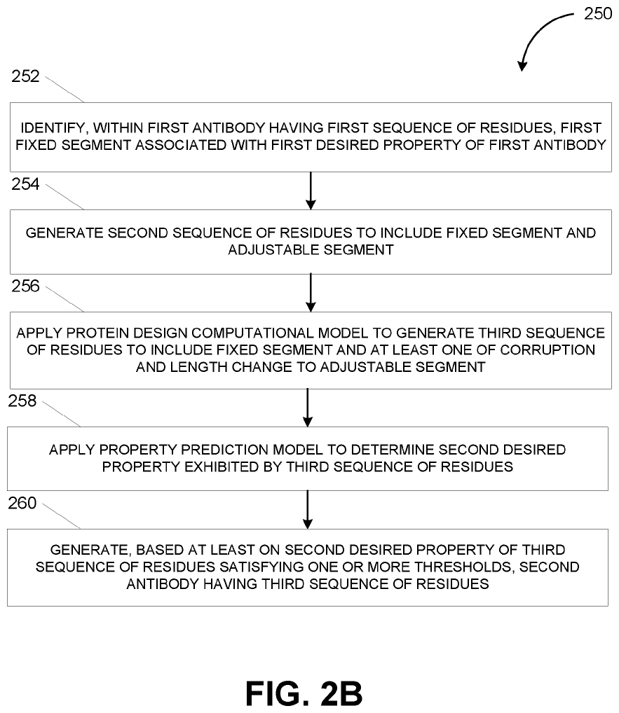

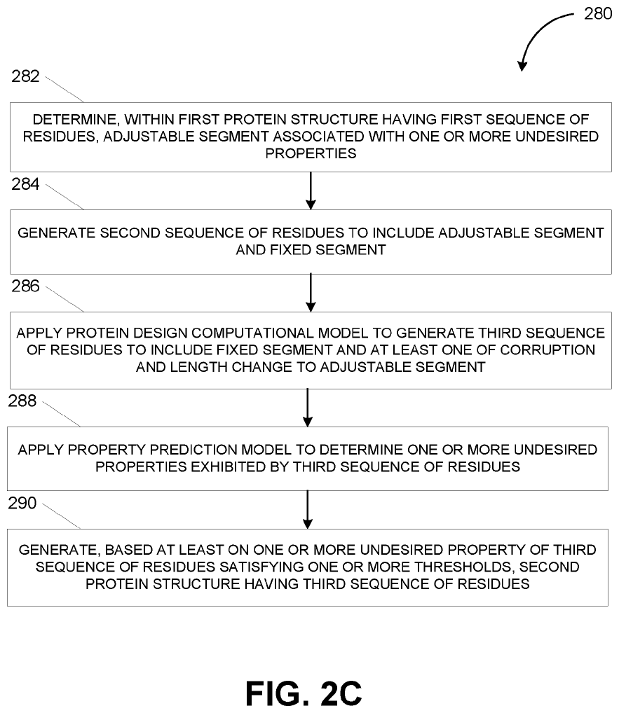

FIG. 2A depicts a flowchart illustrating an example of a process 200 for segment-preserving protein design, in accordance with some example embodiments. Referring to FIGS. 1 and 2A, the process 200 may be performed by the protein design engine 110 to generate one or more protein sequences. For example, in some example embodiments, the protein design engine 110 may apply the protein design computational model 115 to generate, based at least on a first protein sequence having one or more fixed segments, a second protein sequence having the same fixed segments.

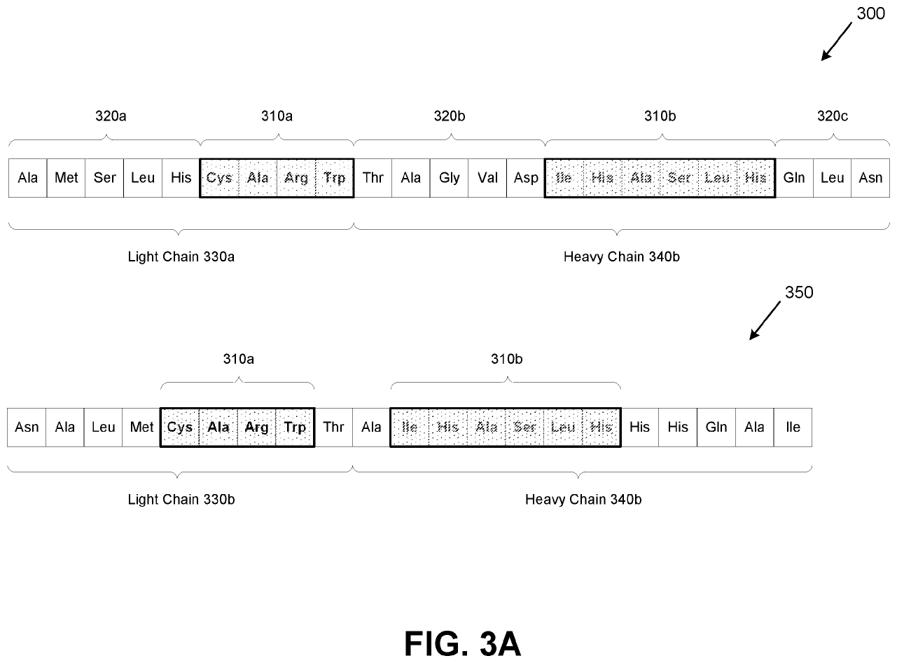

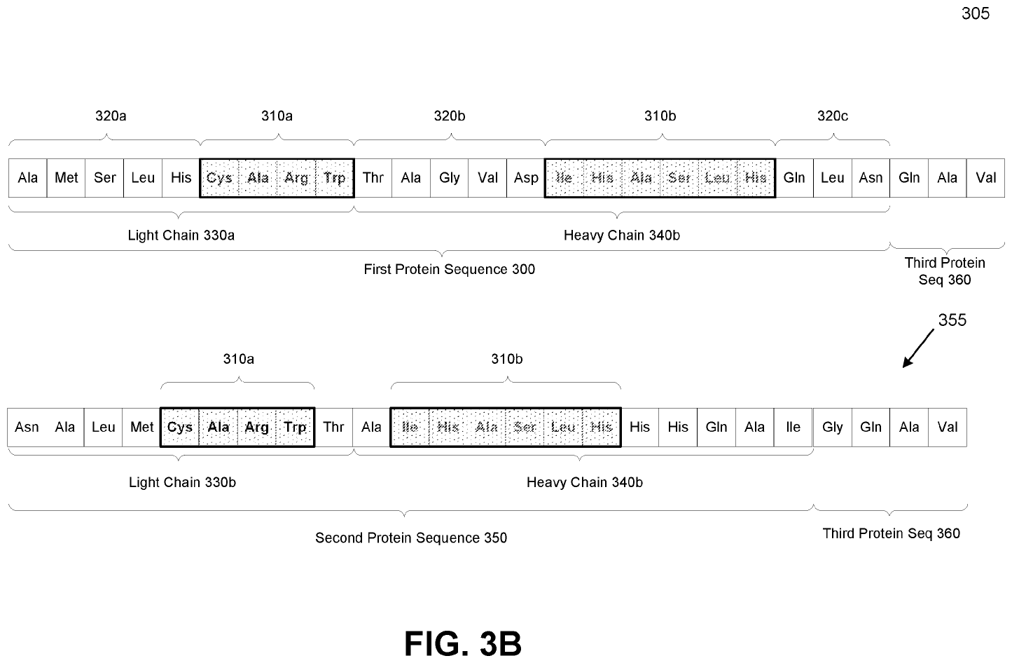

At 202, the protein design engine 110 may determine, with a protein structure having a first sequence of residues, a fixed segment and an adjustable segment. To further illustrate, FIG. 3A depicts a schematic diagram illustrating an example of a first protein sequence 300 corresponding to a first sequence of amino acid residues. As shown in FIG. 3A, the first protein sequence 300 may include a first fixed segment 310a and a second fixed segment 310b, each of which corresponding to sub-sequences of amino acid residues present within the first protein sequence 300. The first fixed segment 310a and the second fixed segment 310b may be associated with one or more desired properties of the first protein sequence 300. Accordingly, as will be described in more detail below, when the protein design engine 110 generates, based on the first protein sequence 300, a second protein sequence 350 corresponding to a second sequence of amino acid residues, the protein design engine 110 may preserve the first fixed segment 310a and the second fixed segment 310b such that first fixed segment 310a and the second fixed segment 310b are also present in the second protein sequence 350. Moreover, as shown in FIG. 3A, the first protein sequence 300 may also include one or more adjustable segments including, for example, a first adjustable segment 320a, a second adjustable segment 320b, and a third adjustable segment 320c. As will be described in more detail below, to generate the second protein sequence 350 while preserving the first fixed segment 310a and the second fixed segment 310b, the protein design engine 110 may modify the first adjustable segment 320a, the second adjustable segment 320b, and/or the third adjustable segments 320c.

At 204, the protein design engine 110 may identify a desired property associated with the protein structure. In some example embodiments, the protein design engine may leverage a priori biological, chemical, and/or physical knowledge to impose certain constraints when generating, for example, the second protein sequence 350 based on the first protein sequence 300. For example, the first protein sequence 300 may be an antibody that exhibits a binding affinity towards a certain antigen (e.g., a viral antigen, a tumor antigen, and/or the like) and/or another desired property such as expression, non-specificity, stability, immunogenicity, human-ness, self-association, and/or the like. Moreover, that desired property may be attributable to certain segments within the first protein sequence 300. Binding affinity, for example, may be associated with the first fixed segment 310a corresponding to a first complementarity determining region (CDR) on a first light chain 330a of the antibody and the second fixed segment 310b corresponding to a second complementarity determining region (CDR) on a first heavy chain 340a of the antibody. Accordingly, when generating the second protein sequence 350 based on the first protein sequence 300, the protein design engine 110 impose certain constraints in order to preserve and, in some cases, enhance, the desired properties exhibited by the first protein sequence 300.

Referring again to FIG. 3A, in cases where the first protein sequence 300 is an antibody, in addition to preserving the first fixed segment 310a and the second fixed segment 310b, preserving the desired property of the first protein sequence 300 may also require keeping the first fixed segment 310a and the second fixed segment 310b within the identical chain in the second protein sequence 350. Accordingly, as will be explained in more detail below, the input provided to the protein design computational model 115 to sample the data distribution may include one or more identifiers (e.g., separator tokens, tags, and/or the like) configured to enable a differentiation between a first portion of the first protein sequence 300 corresponding to the first light chain 330a and a second portion of the first protein sequence 300 corresponding to the first heavy chain 340a. It should be appreciated that the inclusion of such identifiers may be optional in cases where the first protein sequence 300 is a single, monolith sequence without any subunits.

At 206, the protein design engine 110 may use a protein design computational model to generate a second sequence of residues having at least one of a corruption and a length change to the adjustable segment. In some example embodiments, the protein design engine 110 may generate the second protein sequence 350 to also include the same first fixed segment 310a and the second fixed segment 310b as the first protein sequence 300. For example, the protein design engine 110 may modify one or more of the first adjustable segment 320a, the second adjustable segment 320b, and the third adjustable segments 320c by introducing at least one of a corruption (e.g., an insertion, a deletion, and/or a modification of an amino acid residue) and a length change. In doing so, the protein design engine 110 may generate an intermediate sequence having at least one of a corruption and a length change relative to the first protein sequence 300. The protein design engine 110 may decode the intermediate sequence in order to generate the second protein sequence 350. Doing so may preserve at least a portion of the desired properties, such as binding affinity towards certain antigens, exhibited by the first protein sequence 300.

The protein design engine 110 may generate the second protein sequence 350 by applying the protein design computational model 115, which may be implemented as one or more machine learning models (e.g., autoencoders and/or the like). For instance, the protein design computational model 115 may be applied to sample a data distribution learned by the protein design computational model 115 through training. The data distribution may correspond to a reduced dimensional representation of the sequences of residues forming a variety of known protein sequences. In doing so, the protein design engine 110 may identify candidate protein sequences with a high probability of being functional, especially when compared to an indiscriminate exploration of the combinatorial search space of every possible permutation of amino acid residues that can form a protein structure.

In some example embodiments, the sampling of the data distribution includes the protein design computational model 115 generating an encoding of the first protein sequence 300 before decoding an intermediate sequence having at least one of a corruption (e.g., an insertion, a deletion, and/or a modification of an amino acid residue) or a length change relative to the first protein sequence 300. Changes made to the first protein sequence 300 when generating the second protein sequence 350 may be confined to the adjustable segments of the first protein sequence 300. For example, any corruptions made to the first protein sequence 300 are confined to the first adjustable segment 320a, the second adjustable segment 320b, and the third adjustable segments 320c. Furthermore, any length change between the first protein sequence 300 and the second protein sequence 350 may be distributed amongst the first adjustable segment 320a, the second adjustable segment 320b, and/or the third adjustable segments 320c. The length change may be evenly distributed amongst the first adjustable segment 320a, the second adjustable segment 320b, and/or the third adjustable segments 320c, or distributed at varying intervals of the first adjustable segment 320a, the second adjustable segment 320b, and/or the third adjustable segments 320c.

In some example embodiments, the input provided to the protein design computational model 115 may include one or more identifiers to enable a differentiation between different components of the same protein sequence and/or different protein sequences. For example, FIG. 3A depicts one example scenario in which the first protein sequence 300 is an antibody having the first light chain 330a and the first heavy chain 340a. In instances where the first protein sequence 300 is an antibody, the generating of the second protein sequence 350 may be performed based on the one or more identifiers such that a fixed segment present on the heavy chain (or light chain) of the first protein sequence 300 remains on the identical chain in the second protein sequence 350. Accordingly, as shown in FIG. 3A, the first fixed segment 310a from the first light chain 330a of the first protein sequence 300 may remain in a second light chain 330b of the second protein sequence 300 while the second fixed segment 310b from the first heavy chain 340a of the first protein sequence 300 may remain in a second heavy chain 340b of the second protein sequence 350.

FIG. 3B depicts another example scenario in which the first protein sequence 300 is a part of an input sequence 305 that also includes a third protein sequence 360 (e.g., corresponding to an antigen having a certain binding affinity towards the antibody). The presence of the one or more identifiers, which may include separator tokens and tags, may enable the protein design computational model 115 to differentiate between the first light chain 330a of the first protein sequence 300 and the first heavy chain 340a of the first protein sequence 300 as well as between the first protein sequence 300 and the third protein sequence 360. Accordingly, the presence of the one or identifiers may prevent the fixed segments of the first protein sequence 300 from being swapped onto a wrong chain in the second protein sequence 350 and from inadvertently becoming a portion of the third sequence 360. For instance, in the example of the output sequence 355 shown in FIG. 3B, the one or more identifiers present in the input sequence 305 may ensure that the fixed segments present in the first protein sequence 300 remain in the second protein sequence 350 and, more specifically, on the identical chain as in the first protein sequence 300. The identifiers may further be used to collectively analyze protein sequences associated with light chain(s) or collectively analyze protein sequences associated with heavy chain(s).

The protein design engine 110 may perform multiple sampling iterations, with each sampling iteration identifying at least one candidate protein sequence. Examples of techniques to iteratively sample from the data distribution includes a Markov Chain Monte Carlo (MCMC), importance sampling (IS), rejection sampling, Metropolis-Hastings, Gibbs sampling, slice sampling, exact sampling, and/or the like. Candidate protein sequences may be subjected to further functional and/or structural analysis to determine, for example, whether each candidate protein sequence exhibits a desired property. For example, as shown in FIG. 1, the analysis controller 120 may analyze the second protein sequence 350 generated by the protein design engine 110 by applying one or more of the property prediction model 122 (e.g., to evaluate one or more properties of the second protein sequence 350), the structural modeling engine 124 (e.g., to determine a secondary structure and/or a tertiary structure of the second protein sequence 350), and molecular dynamics simulator 126 (e.g., to determine an energy state and stability of the second protein sequence 350). At least a portion of the results associated with the sampling, the functional analysis, and/or the structural analysis may be provided for display, for example, in the user interface 135 at the client device 130.

At 208, the protein design engine 110 may use the protein design computational model to generate a modified protein structure having the second sequence of residues. In some example embodiments, a modified protein structure corresponding to the second protein sequence 350 may be generated in silico upon satisfaction of one or more conditions. As noted, the protein design engine 110 may continue to sample the data distribution until one or more conditions are satisfied including, for example, the identification of a threshold quantity of candidate protein sequences, the identification of a threshold quantity of protein sequences exhibiting a desired property, and/or the like. In cases where a candidate protein sequence, such as the second protein sequence 350, is determined to exhibit certain desired properties, such as a binding affinity towards certain antigens, the protein design engine 110 may identify the second protein sequence 350 as a modified protein structure that is suitable for further in vitro analysis and/or in vivo characterization.

As noted, in some example embodiments, the generating of the second protein sequence 350, which includes encoding and decoding an intermediate sequence corresponding to the first protein sequence 300, may be performed based on the one or more identifiers such that a fixed segment present in the first protein sequence 300 (e.g., on the heavy chain (or light chain) of the antibody corresponding to the first protein sequence 300) remains on the identical chain in the second protein sequence 350. For example, as shown in FIG. 3A, the first fixed segment 310a from the first light chain 330a of the first protein sequence 300 may remain in the second light chain 330b of the second protein sequence 300 while the second fixed segment 310b from the first heavy chain 340a of the first protein sequence 300 may remain in the second heavy chain 340b of the second protein sequence 350.

In some example embodiments, the one or more identifiers may enable a differentiation between multiple components present within the input sequence provided to the protein design computational model 115 including, for example, subunits within a single protein sequence (e.g., light chain and heavy chain), separate protein sequences, and/or the like. For example, where the first protein sequence 300 is an antibody, an input including the first protein sequence 300 may include one or more additional protein sequences corresponding to antigens that have a certain binding affinity towards the antibody. Accordingly, the input including the first protein sequence 300 may include one or more identifiers (e.g., separator token, tags, and/or the like) to enable a differentiation between the first light chain 330a and the first heavy chain 340a of the first protein sequence 300. Furthermore, the input including the first protein sequence 300 may include one or more additional identifiers to enable a differentiation between the first protein sequence 300 and the additional protein sequences.

To further illustrate, FIG. 3B depicts an example of the input sequence 305 including the first protein sequence 300 and the output sequence 355 including the second protein sequence 350. As noted, in cases where the first protein sequence 300 is an antibody, the additional protein sequences present in the input including the first protein sequence 300 may correspond antigens exhibiting a certain binding affinity towards the antibody. In the example shown in FIG. 3B, the input sequence 305 includes the first protein sequence 300 and the third protein sequence 360 corresponding to, for example, an antigen having a certain binding affinity towards the antibody. Moreover, the protein design engine 110 may generate, based at least on the input sequence 305, the output sequence 355 including the second protein sequence 350 and the third protein sequence 360.

As noted, the first protein sequence 300 may include one or more fixed segments (e.g., the first fixed segment 310a, the second fixed segment 310b, and/or the like), which are preserved when generating the output sequence 355 such that the second protein sequence 355 includes the same fixed segments as the first protein sequence 350. The output sequence 355 may be generated based on the one or more identifiers present in the input sequence 305 (e.g., separator tokens tags, and/or the like) such that the fixed segments present in the first protein sequence 300 remain in the second protein sequence 350 and, more specifically, on the identical chain as in the first protein sequence 300. Absent the identifiers, the fixed segments of the first protein sequence 300 may be swapped onto a wrong chain in the second protein sequence 350 or inadvertently become a portion of the third sequence 360.

In some example embodiments, the third protein sequence 360 may be a fixed segment such that the protein design engine 110 is able to evolve at least a portion of the first protein sequence 300 without also modifying the third protein sequence 360. In cases where the first protein sequence 300 corresponds to an antibody and the third protein sequence 350 corresponds to an antigen exhibiting a certain binding affinity towards the antibody, this may be tantamount to evolving the antibody while keeping the antigen immutable. Alternatively, at least a portion of the third protein sequence 360 may be an adjustable segment, in which case the protein design engine 110 may be able to evolve at least a portion of the third protein sequence 360 while also modifying the first protein sequence 300. Referring again to the scenario where the first protein sequence 300 corresponds to an antibody and the third protein sequence 350 corresponds to an antigen having a certain binding affinity towards the antibody, this may tantamount to evolving the antibody along with the antigen.

Referring again to FIG. 1, as noted, the protein design engine 110 may apply the protein design computational model 115 in order to generate, based on the first protein sequence 300, the second protein sequence 350. Furthermore, as noted, the protein design computational model 115 may be implemented as an autoencoder (e.g., a denoising autoencoder (DAE) and/or the like), which generates the second protein sequence 350 by sampling a data distribution corresponding to a reduced dimension representation (e.g., a manifold and/or the like) of a variety of known protein sequences. The sampling of the data distribution may include encoding the first protein sequence 300 before decoding an intermediate sequence having at least one of a corruption (e.g., an insertion, a deletion, and/or a modification of an amino acid residue) or a length change relative to the first protein sequence 300 to generate the second protein sequence 350.

In some example embodiments, when implemented as an autoencoder (e.g., a denoising autoencoder (DAE) and/or the like, the protein design computational model 115 may include a corruption process C (\tilde{x}|x), an encoder F, and a decoder G. Moreover, the protein design computational model 115 may include a length converter, which may be implemented as a classifier configured to determine, based on a probability distribution of possible length differences between the first protein sequence 300 and the second protein sequence 350, a length difference between the first protein sequence 300 and the second protein sequence 350. In order to preserve the first fixed segment 310a and the second preserved segment 310b, the length difference between the first protein sequence 300 and the second protein sequence 350 may be distributed amongst one or more of the first adjustable segment 320a, the second adjustable segment 320b, and the third adjustable segment 320c. As will be described in more details below, in instances where the protein design computational model 115 ingests a fixed-length representation of the first protein sequence 300, the protein design computational model 115 may be implemented without the length converter. Instead of the length converter, the length difference between the first protein sequence 300 and the second protein sequence 350 may be achieved through the addition and/or deletion of gap characters (e.g., ghost residues and/or the like) from one or more adjustable segments of the first protein sequence 300. Accordingly, in cases where the protein design computational model 115 ingests the fixed-length representation of the first protein sequence 300, the

Alternatively, where the protein design computational model 115 ingests a fixed-length representation of the first protein sequence 300 (e.g., generated by the encoder 113), the length changes, which are confined to one or more of the first adjustable segment 320a, the second adjustable segment 320b, and the third adjustable segment 320c, may be accomplished by inserting and/or deleting one or more amino acid residues in the first protein sequence 300. For example, in some cases, an amino acid residue may be inserted to increase the length of the first protein sequence 300 by at least replacing, with the amino acid residue, a gap character (e.g., ghost residue and/or the like) in the one or more adjustable segments 320 in the fixed length representation of the first protein sequence 300. The length of the first protein sequence 300 may be decreased by deleting an amino acid residue from the fixed-length representation of the first protein sequence 300, which includes replacing the amino acid residue in the one or more adjustable segments in the fixed length representation of the first protein sequence 300 with a gap character (e.g., a ghost residue and/or the like).

The protein design computational model 115 may operate on a sequence of discrete tokens, x=(x₁, x₂, . . . , x_{L}), wherein each token xₜ is an item from a finite vocabulary V. When applied towards protein design, the vocabulary V may include the amino acid residues that be present in a protein sequence. The sequence x is corrupted with the corruption process C, resulting in a corrupted sequence \tilde{x}˜C (\tilde{x}|x). The corruption process C associated with the protein design computational model 115 can be arbitrary as long as it is largely local and unstructured. In some cases, the corruption process C may even alter the length of the sequence such that |x|≠|\tilde{x}|.

The encoder F can be implemented using a variety of deep learning architectures including, for example, transformers, convolutional neural networks, recurrent neural networks, and/or the like. The encoder F turns the corrupted sequence \tilde{x} into a set of hidden vectors, h= (h₁, h₂, . . . , h_{|\tilde{x}|})), wherein each hidden vector hₜ ∈ R^{d}. That is, each hidden vector h may correspond to one of the amino acid residues in the corrupted sequence \tilde{x}. The hidden vectors h may then be pooled to form a single-vector representation

$$\underline{h} = \frac{1}{\vert \tilde{x} \vert} {\sum }_{t = 1}^{\vert \tilde{x} \vert} {h}_{t} .$$

This pooled single-vector representation h is used by the length converter to predict the change in length between the first protein sequence 300 and the second protein sequence 350.

In some cases, the length converter may be a machine learning model that is trained to output a predicted length change Δl where Δl*=|\tilde{x}|−|x|. When the trained machine learning model samples from the data distribution during inference time, the predicted length change Δl may be applied to adjust the size of the hidden vector set h with the adjusted hidden vector set having an |\tilde{x}|+Δl number of hidden vectors, thus generating a transformed hidden vector sequence z=(z₁, . . . , z_{|\tilde{x}|}+Δl), wherein zₜ=Σ_{t′=1}^{|\tilde{x}|}ω_{t,t}hₜ, with the position-based softmax weights ω_{t,t}, preferring hₜ, closest to the length-scaled position

$$\frac{\vert \tilde{x} \vert}{( \vert \tilde{x} \vert + Δ l ) t} as {ω}_{t , t ′}^{t} = \frac{exp ( {q}_{t , {t}^{′}} )}{{\sum }_{{t}^{″} = 1}^{\vert \tilde{x} \vert} exp ( {q}_{t , {t}^{″}} )} and {q}_{t , t ′} ∝ \frac{1}{2 {σ}^{2}} {( {t}^{′} - \frac{\vert \tilde{x} \vert}{\vert \tilde{x} \vert + Δ l} t )}^{2} .$$

That is, the transformed vector sequence z may include a quantity of hidden vectors h as adjusted by the length change Δl. For example, in cases where the length change Δl increases the quantity of amino acid residues, the transformed vector sequence z may include a corresponding Δl more quantity of hidden vectors h. Contrastingly, where the length change Δl reduces the quantity of amino acid residues, the transformed vector sequence z may include a corresponding Δl fewer quantity of hidden vectors h.

The decoder G then takes this transformed hidden vector sequence z and outputs a corresponding sequence of logit vectors, \tilde{y}=(\tilde{y}₁, . . . , \tilde{y}_{|\tilde{x}|}+Δl), wherein each logit vector \tilde{y}ₜ ∈R^{|v|}. These logit vectors y can be turned into probability distributions over the vocabulary V of different amino acid residues in many different ways. That is, each logit vector \tilde{y}ₜ ∈R^{|v|} may be turned into a probability distribution across the different amino acid residues that may occupy the corresponding position. For example, each logit vector \tilde{y}ₜ ∈R^{|v|} may be turned into a probability distribution that includes, for each of the twenty possible types of amino acid residues, a probability that the corresponding position is occupied by that amino acid residue.

One example technique for transforming the logit vectors \tilde{y} is a non-autoregressive approach in which each logit vector \tilde{y}ₜ is turned independently into a distribution

$$p ( {y}_{t} = v \vert \tilde{x} , Δ l ) = \frac{exp ( {\tilde{y}}_{t}^{v} + {b}^{v} )}{{\sum }_{v ′ ∈ V} exp ( {\tilde{y}}_{{t}^{}}^{v ′} + {b}^{v ′} )} ,$$

wherein b^{v} denotes a bias for the token v. Alternative techniques for turning the logit vectors \tilde{y} into probability distributions over the vocabulary V include conditional random fields, autoregressive modeling, and/or the like.

During training of the protein design computational model 115, the encoder F may be trained to generate, based on a corrupted version of the first protein sequence 300, an encoding of the first protein sequence 300 that enables the decoder G to generate a decoding that exhibits a minimal difference relative to the original, uncorrupted version of the first protein sequence 300. That is, during training, the encoder F and the decoder G may be trained by minimizing the negative log-probability of the original sequence x given the corrupted version \tilde{x} and a known length change Δl*, while the negative log-probability of the known length change Δl* is applied towards training the length converter. Once training of the protein design computational model 115 is complete, one or more candidate protein sequences may be drawn from the protein design computational model 115, for example, by repeating the process of corruption, length conversion, and reconstruction.

As noted, to generate the second protein sequence 350 to exhibit one or more of the same desired properties exhibited by the first protein sequence 300, the protein design engine 110 may preserve one or more fixed segments associated with the desired properties such as, for example, the first fixed segment 310a, the second fixed segment 310b, and/or the like. Thus, if x=(x₁, x₂, . . . , x_{L}) corresponds to the first protein sequence 300 serving as the basis for drawing a series of candidate protein sequences from the trained protein design computational model 115, the first protein sequence 300 may include a set of non-overlapping segments (e.g., sub-sequences of amino acid sequences) that are preserved in each of the candidate protein sequences drawn from the trained protein design computational model 115. This set of non-overlapping segments may be denoted as s=((i₁,j₁), . . . , (i_{K},j_{K})), subject to iₖ≤jₖ for all values of k and jₖ<iₖ, for all k′>k. This set of non-overlapping segments may be referred to as a fixed-segment set whereas the complement segment set s may include the other segments within the first protein sequence 300 that can be modified to generate the candidate protein sequences. The complement segment set s may be referred to as the adjustable-segment set and denoted as s=((0, i₁−1), (j₁+1,i₂−1), . . . , (j_{K}+1, |x|+1)).

In order to preserve the fixed segments swhile altering the adjustable segments s, including the lengths at least some adjustable segments s, the corruption process C may be configured to avoid corrupting the fixed segments s. For example, instead of inserting, deleting, or modifying amino acid residues from arbitrary portions of the first protein sequence 300, the corruption process C may limit these corruptions to the adjustable segments swhile avoiding the fixed segments s. Doing so generates a corrupted sequence \tilde{x} and changes the segment set s in order to appropriately reflect the changes in the indices due to insertions and deletions. As used herein, \tilde{s} may denote the fixed segment set present in the corrupted sequence \tilde{x}.

The encoder F encodes the corrupted sequence x to generate a hidden vector set h that corresponds to the corrupted sequence \tilde{x}. Meanwhile, in order to prevent altering the length of any fixed segments s, any length change Δl determined by the length converter may be distributed amongst the adjustable segments s in the first protein sequence 300 in a variety of ways. One example is to distribute the predicted length change Δl proportional to the original lengths of the adjustable segments s. That is, the predicted length change Δl may be applied towards increasing (or decreasing) the length of one or more adjustable segments s such that

$$( {\tilde{J}}_{k} + 1 , {\tilde{l}}_{k + 1} - 1 ) and ⌈ \frac{( {\tilde{l}}_{k + 1} - {\tilde{J}}_{k} + 1 )}{\begin{matrix}{\sum }_{{k}^{′} = - 1}^{K} & ( {\tilde{l}}_{k + 1} - {\tilde{J}}_{k} + 1 )\end{matrix}} Δ l ⌉ ,$$

wherein \tilde{J}₀=0 and \hat{ι}_{K+1}=|\tilde{x}|+1.

Upon distributing the length difference Δl amongst the adjustable segments s, the protein design engine 110 may construct an index map o mapping the segments in the resulting intermediate sequence to the corresponding fixed segments s in the corrupted sequence \tilde{x} (e.g., y_{o(\hat{ι}}_{ₖ}_{):o(\tilde{J}}_{ₖ}₎=\tilde{x}_{\tilde{ι}}_{ₖ}_{:\tilde{J}}_{ₖ}₎. Here, o(\tilde{s}) may denote the fixed-segment set derived from s and the distribution of length change Δl described above.

Length conversion, whether with or without preserving fixed segments, may proceed in accordance with zₜ=Σ_{t′=1}^{|\tilde{x}|}ω_{t,t}hₜ. To enable the preservation of certain fixed segments within the first protein sequence 300, the original hidden vector hₜ of a token within a fixed segment s may be carried over within the fixed segment s in order to provide the decoder G a hint about the fixed segments s and their contents. This operation is expressed by Equation (1) below:

$${z}_{t} ← \{ \begin{matrix}( 1 - β ) {z}_{t} + β {h}_{{o}^{- 1} ( t )} , if t ∈ o ( \tilde{s} ) {z}_{t} , \\ {z}_{t} , if t ∉ o ( \tilde{s} )\end{matrix} \qquad ( 1 )$$

wherein o⁻¹ denotes the inverse index map, and β∈[0, 1] denotes the strength of carry-over. That is, for a token t that is within a fixed segment s, Equation (1) outputs the original hidden vector hₜ. Contrastingly, in instances where the token t is not within a fixed segment s, Equation (1) outputs the transformed hidden vector.

Alternatively, to force the modification of certain residues within an adjustable segment s of the first protein sequence 300, the negative value of the original hidden vector hₜ of a token within an adjustable segment s may be carried over within the variable segment s in order to provide the decoder G a hint about the residues that require modification.

The decoder G turns this length-converted and segment-preserving hidden sequence z into a sequence of logit vectors \tilde{y}, which are then modified corresponding to a token with a fixed segment to force the sampled outcome to preserve the token identity as indicated by Equation (2) below:

$${\tilde{y}}_{t}^{v} ← \{ \begin{matrix}∞ , if t ∈ o ( \tilde{s} ) and v = {\tilde{x}}_{{o}^{- 1} ( t )} \\ - ∞ , if t ∈ o ( \tilde{s} ) and v \neq {\tilde{x}}_{{o}^{- 1} ( t )} \\ {\tilde{y}}_{t}^{v} , if t ∉ o ( \tilde{s} )\end{matrix} \qquad ( 2 )$$

In the case non-autoregressive modeling is used to transform each logit vector \tilde{y}ₜ independently into a distribution

$$p ( {y}_{t} = v \vert \tilde{x} , Δ l ) = \frac{exp ( {\tilde{y}}_{t}^{v} + {b}^{v} )}{{\sum }_{v ′ ∈ V} exp ( {\tilde{y}}_{{t}^{}}^{v ′} + {b}^{v ′} )}$$

over the vocabulary V, Equation (2) above would generate a categorical distribution in which a fixed token is assigned the entire probability mass (e.g., 1) to the original token identity. That is, for a token t that is within a fixed segment s, Equation (2) would assign a probability of one to the original type of amino acid residue and a probability of zero to all other types of amino acid residues. Contrastingly, if a conditional random field is used to transform the logit vectors \tilde{y}ₜ into probability distributions over the vocabulary V, application of Equation (2) would prevent any sequence that violates segment preservation constraints from being decoded with non-zero probability.

Where the adjustable segment s includes a residue designated for modification, non-autoregressive modeling may transform the corresponding logit vector \tilde{y}ₜ of the corresponding token into a distribution

$$p ( {y}_{t} = v \vert \tilde{x} , Δ l ) = \frac{exp ( {\tilde{y}}_{t}^{v} + {b}^{v} )}{{\sum }_{v ′ ∈ V} exp ( {\tilde{y}}_{{t}^{}}^{v ′} + {b}^{v ′} )}$$

over the vocabulary V where the original token identity of the residue is assigned a null probability (e.g., 0). That is, Equation (2) would assign a probability of zero to the original type of amino acid residue for a token t that is designated for modification. In cases where a conditional random field is used to transform the logit vector \tilde{y}ₜ of the token into probability distributions over the vocabulary V, any sequence in which the residue designated for modification remains the same would be prevented from being decoded with non-zero probability.

As noted, the sampling of the data distribution associated with the trained protein design computational model 115, which includes the aforementioned corruption, length conversion, and reconstruction, may be repeated iteratively to draw multiple candidate protein sequence segments. The candidate protein sequences may undergo, individually or in groups, subsequent functional and/or structural analysis. For example, referring back to FIG. 1, the analysis controller 120 may analyze one or more candidate protein sequence by applying one or more of the property prediction model 122 (e.g., to evaluate one or more properties of the second protein sequence), the structural modeling engine 124 (e.g., to determine a secondary structure and/or a tertiary structure of the second protein sequence), and the molecular dynamics simulator 126 (e.g., to determine an energy state and stability of the second protein sequence). Moreover, at least a portion of the results associated with the sampling, the functional analysis, and/or the structural analysis may be provided for display, for example, in the user interface 135 at the client device 130.

FIG. 2B depicts a flowchart illustrating another example of a process 250 for segment-preserving protein design, in accordance with some example embodiments. Referring to FIGS. 1 and 2B, the process 250 may be performed by the protein design engine 110 to generate one or more protein sequences. For example, in some example embodiments, the protein design engine 110 may apply the protein design computational model 115 to generate, based at least on a first protein sequence having one or more fixed segments, a second protein sequence having the same fixed segments. In some cases, the protein design engine 110 may apply the protein design computational model 115 to generate the second protein sequence by applying, to the first protein sequence, one or more modifications that preserve a first desired property of the first protein sequence (e.g., binding affinity) while also increasing (or maximizing) a second desired property (e.g., human-ness) of the first protein sequence. For instance, in cases where the first protein sequence is a non-human antibody (e.g., an antibody originating from a non-human species), the process 250 may be performed to humanize the first protein sequence such that the resulting second protein sequence exhibits the same desired properties as the first protein sequence but also sufficient human identity to avoid an immunogenic response in human recipients of a drug formulated with the second protein sequence.

At 252, the protein design engine 110 may identify, within a first antibody having a first sequence of residues, a fixed segment associated with a first desired property of the first antibody. In some example embodiments, the protein design engine 110 may identify, within the first antibody having the first sequence of residues, one or more fixed segments associated with one or more desired properties of the first antibody. For example, in some cases, the first antibody having the first sequence of residues may be a non-human antibody originating from a non-human the protein design engine 110. The protein design engine 110 may identify, within the first sequence of residues, one or more fixed segments (e.g., one or more sub-sequences) corresponding one or more complementarity determining regions (CDRs) of the first antibody. Alternatively and/or additionally, the protein design engine 110 may identify, within the first sequence of residues, one or more fixed segments (e.g., one or more sub-sequences) corresponding to one or more Vernier zone residues present in the first antibody. The one or more complementarity determining regions (CDRs) and/or the Vernier zone residues of the first antibody may be designated as fixed segments such that antibodies are generated to include the same complementarity determining regions (CDRs) and/or Vernier zone residues, thereby preserving the desired properties (e.g., binding affinity towards certain target molecules) associated with these complementarity determining regions (CDRs). In the case of Vernier zone residues, those are amino acid residues located in the framework region of the first antibody and underlie the complementarity determining regions (CDRs). Accordingly, one or more Vernier zone residues may be designated as fixed segments at least because Vernier zone residues could potentially affect the conformation of complementarity determining region (CDR) loop structures and in turn the binding affinity of the corresponding antibody.

At 254, the protein design engine 110 may generate a second sequence of residues to include the fixed segment and an adjustable segment. In some example embodiments, the protein design engine 110 may generate a second sequence of residues to include one or more fixed segments, such as the one or more complementarity determining regions (CDRs), from the first sequence of residues forming the first antibody. The second sequence of residues may be further generated to include one or more adjustable segments, which are modified when one or more antibodies are generated based on the second sequence of residues. For example, in some cases, the one or more adjustable segments may include one or more randomly generated sequences of amino acid residues. Alternatively and/or additionally, the one or more adjustable segments may include one or more known or predetermined sequences of amino acid residues. For instance, in some cases, the one or more adjustable segments may correspond to one or more framework regions of a human antibody, in which case the second sequence of residues may be generated by grafting the one or more fixed segments corresponding to the one or more complementarity determining regions (CDRs) of the non-human antibody onto a human germline framework (e.g., one or more framework regions of a human antibody excluding one or more Vernier zone residues). In this context, the grafting of a first complementarity determining region (CDR) of the non-human antibody onto the human germline framework may be achieved by at least replacing a second complementarity determining region (CDR) of the human antibody with the first complementarity determining region (CDR) of the non-human antibody. Alternatively and/or additionally, a first Vernier zone residue of the non-human antibody may be granted onto the human germline framework by at least replacing a second Vernier zone residue of the human antibody with the first Vernier zone residue of the non-human antibody. Accordingly, in some cases, the resulting second sequence of residues may include one or more fixed segments corresponding to one or more complementarity determining regions (CDRs) and/or Vernier zone residues of the non-human antibody and one or more framework regions (FRs) (excluding one or more Vernier zone residues) of the human antibody.

At 256, the protein design engine 110 may apply the protein design computational model 115 to generate a third sequence of residues to include the fixed segment and at least one of a corruption and a length change to the adjustable segment. In some example embodiments, the protein design engine 110 may apply the protein design computation model 115 to generate a third sequence of residues by at least modifying the one or more adjustable segments in the second sequence of residues while keeping the one or more fixed segments in the second sequence of residues the same. Accordingly, the resulting third sequence of residues may include the same fixed segments as the second sequence of residues while the adjustable segments from the second sequence of residues may have undergone at least one of a corruption (e.g., an insertion, a deletion, and/or a modification of an amino acid residue) and a length change. In instances where the one or more fixed segments correspond to the one or more complementarity determining regions (CDRs) and/or Vernier zone residues of the non-human antibody, the third sequence of residues may be generated to include the same complementarity determining regions (CDRs) and/or Vernier zone residues as the non-human antibody such that a second antibody having the third sequence of residues may exhibit the same desired properties (e.g., binding affinity towards certain target molecules) as the non-human antibody. Moreover, where the one or more adjustable segments correspond to one or more randomly generated sequences of amino acid residues and/or the one or more framework regions of the human antibody, the third sequences of residues may be generated to include changes to the adjustable segments that optimize certain desired properties (e.g., increase the human-ness) of the resulting second antibody as well as render these adjustable segments more compatible with the one or more fixed segments (e.g., the one or more complementarity determining regions (CDRs)) in the second antibody.

At 258, the protein design engine 110 may apply the property prediction model 122 to determine a second desired property exhibited by the third sequence of residues. In some example embodiments, the protein design engine 110 may apply the property prediction model 122 to determine one or more properties of the third sequence of residues having the same complementarity determining regions (CDRs) and/or Vernier zone residues as the non-human antibody. For example, in instances where the protein design computation model 115 is trained to modify the adjustable segments to increase the human-ness of the resulting sequence of residues, the property prediction model 122 may be applied to determine the human-ness of the third sequence of residues. Additionally, in some cases, the property prediction model 122 may also be applied to determine whether the third sequence of residues maintains the same desired property associated with the fixed segments included in the third sequence of residues. For instance, in some cases, the protein design engine 110 may apply the property prediction model 122 to determine whether the third sequence of residues exhibits the binding affinity associated with one or more complementarity determining regions (CDR) and/or Vernier zone residues from the non-human antibody.

At 260, the protein design engine 110 may generate, based at least on the second desired property of the third sequence of residues satisfying one or more thresholds, a second antibody having the third sequence of residues. In some example embodiments, the protein design engine 110 may generate a second antibody having the third sequence of residues if the output of the property prediction model 122 indicates that the third sequence of residues exhibits one or more desired properties. For example, the protein design engine 110 may identify a second antibody having the third sequence of residues as a candidate for synthesis and further testing (e.g., in vitro analysis, in vitro characterization, and/or the like) if the output of the property prediction model 122 indicates that the second sequence of residues exhibit sufficient human-ness and, in some cases, binding affinity towards certain target molecules.

FIG. 2C depicts a flowchart illustrating another example of a process 280 for segment-preserving protein design, in accordance with some example embodiments. Referring to FIGS. 1 and 2C, the process 280 may be performed by the protein design engine 110 to generate one or more protein sequences. For example, in some example embodiments, the protein design engine 110 may apply the protein design computational model 115 to generate, based at least on a first protein sequence having one or more fixed segments, a second protein sequence having the same fixed segments. In some cases, the protein design engine 110 may apply the protein design computational model 115 to generate the second protein sequence by applying, to the first protein sequence, one or more modifications that reduce (or minimize) one or more undesired properties of the first protein sequence while preserving one or more desired properties of the first protein sequence. For instance, the one or more modifications may include altering and/or removing one or more residues (or patterns of adjacent and/or non-adjacent residues) within one or more adjustable segments of the first protein sequence while preserving one or more fixed segments identified as being associated with the one or more desired properties.

At 282, the protein design engine 110 may determine, within a first protein structure having a first sequence of residues, an adjustable segment associated with one or more undesired properties. In some example embodiments, the protein design engine 110 may identify, within a first protein structure having a first sequence of residues, one or more adjustable segments that are associated with undesired properties. In some cases, the one or more adjustable segments may include one or more specific amino acid residues or pattern of amino acid residues (e.g., motifs), including those formed by adjacent as well as non-adjacent amino acid residues, that are associated with certain undesired properties. For example, tryptophan residues may be prone to oxidization under chemical stress, “NP” motifs may be prone to chemical modification by protease enzymes, and aspartate residues may be prone to chemical isomerization while in formulation. Accordingly, as described in more detail below, these residues (or residue patterns) may form at least a portion of the adjustable segments identified within the first sequence of residues. Moreover, in some cases, these residues (or residue patterns) may be designated for modification, meaning that changes made to the adjustable segments of the first sequence of residues are required to include changes to these residues (or residue patterns) such that these residues (or residue patterns) gare absent from a second sequence of residue generated based on the first sequence of residues.

At 284, the protein design engine 110 may generate a second sequence of residues to include the adjustable segment and a fixed segment. In some example embodiments, in addition to the adjustable segment containing one or more amino acid residues (or patterns of amino acid residues) associated with one or more undesired properties, the protein design engine 110 may generate the second sequence of residues to include one or more fixed segment associated with the one or more desired properties. In the aforementioned antibody example, the one or more fixed segments may include a complementarity determining region (CDR) and/or one or more Vernier zone residues of an antibody, which are associated with the binding affinity of the antibody. Meanwhile, the one or more adjustable segments may include one or more framework regions (FRs) of an antibody. Alternatively and/or additionally, the one or more adjustable segments may include one or more randomly generated sequences of amino acid residues.

At 286, the protein design engine 110 may apply the protein design computational model 115 to generate a third sequence of residues to include the fixed segment and at least one of a corruption and a length change to the adjustable segment. In some example embodiments, the protein design engine 110 may apply the protein design computational model 115 to generate, based at least on the second sequence of residues, the third sequence of residues to include the one or more fixed segments and the one or more adjustable segments modified with at least one of a corruption (e.g., an insertion, a deletion, and/or a modification of an amino acid residue) and a length change. In some cases, in addition to ensuring that the changes made to the second sequence of residues replace (and/or remove) the one or more amino acid residues (or patterns of amino acid residues) associated with the one or more undesired properties, the protein design computational model 115 may avoid making any modifications to the one or more fixed segments of the second sequence of residues in order to preserve the desired properties associated with these fixed segments. Furthermore, when editing the one or more adjustable segments of the second sequence of residues, the protein design computational model 115 may ensure that the changes made to the one or more adjustable segments of the second sequence of residues reduce (or minimize) the undesired properties, increase (or maximize) the desired properties, as well as increase (or maximize) the compatibility between the adjustable segments and the fixed segments of the resulting third sequence of residues.

At 288, the protein design engine 110 may apply the property prediction model 122 to determine the one or more undesired properties of the third sequence of residues. For example, in some cases, the protein design engine 110 may apply the property prediction model 122 to determine the one or more undesired properties present in the third sequence of residues, which has been generated by at least replacing (and/or removing) one or more residues and/or patterns of residues associated with one or more undesired properties. In addition, in some cases, the protein design engine 110 may also apply the property prediction model 122 to determine the one or more desired properties exhibited by the third sequence of residues, which has been generated to preserve the one or more fixed segments associated with the one or more desired properties.

At 290, the protein design engine 110 may generate, based at least on the one or more undesired property of the third sequence of residues satisfying one or more thresholds, a second protein structure having the third sequence of residues. In some example embodiments, the protein design engine 110 may generate a second antibody having the third sequence of residues if the output of the property prediction model 122 indicates that the third sequence of residues exhibits one or more desired properties but not the one or more undesired properties. For example, the protein design engine 110 may identify a second antibody having the third sequence of residues as a candidate for synthesis and further testing (e.g., in vitro analysis, in vitro characterization, and/or the like) if the output of the property prediction model 122 indicates that the second sequence of residues exhibits sufficient binding affinity to a target molecule, human-ness, expression, thermostability, and/or viscosity but lacks a propensity for oxidation, chemical modification, and/or chemical isomerization.

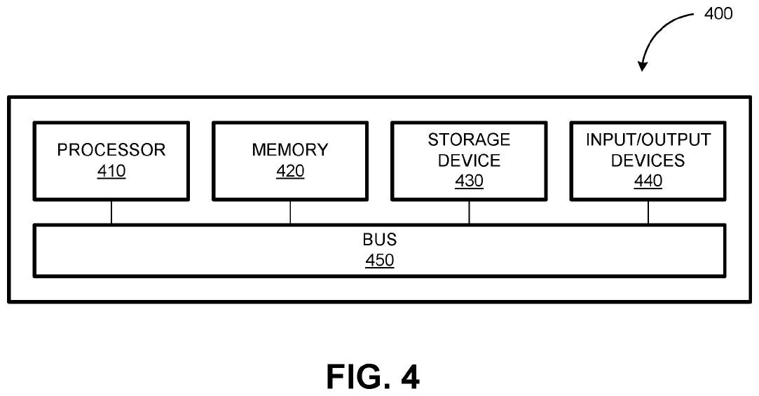

FIG. 4 depicts a block diagram illustrating an example of computing system 400, in accordance with some example embodiments. Referring to FIGS. 1-4, the computing system 400 may be used to implement the protein design engine 110, the analysis controller 120, the client device 130, and/or any components therein.

As shown in FIG. 4, the computing system 400 can include a processor 410, a memory 420, a storage device 430, and input/output devices 440. The processor 410, the memory 420, the storage device 430, and the input/output devices 440 can be interconnected via a system bus 450. The processor 410 is capable of processing instructions for execution within the computing system 400. Such executed instructions can implement one or more components of, for example, the protein design engine 110, the analysis controller 120, the client device 130, and/or the like. In some example embodiments, the processor 410 can be a single-threaded processor. Alternately, the processor 410 can be a multi-threaded processor. The processor 410 is capable of processing instructions stored in the memory 420 and/or on the storage device 430 to display graphical information for a user interface provided via the input/output device 440.

The memory 420 is a computer readable medium such as volatile or non-volatile that stores information within the computing system 400. The memory 420 can store data structures representing configuration object databases, for example. The storage device 430 is capable of providing persistent storage for the computing system 400. The storage device 430 can be a floppy disk device, a hard disk device, an optical disk device, or a tape device, or other suitable persistent storage means. The input/output device 440 provides input/output operations for the computing system 400. In some example embodiments, the input/output device 440 includes a keyboard and/or pointing device. In various implementations, the input/output device 440 includes a display unit for displaying graphical user interfaces.

According to some example embodiments, the input/output device 440 can provide input/output operations for a network device. For example, the input/output device 440 can include Ethernet ports or other networking ports to communicate with one or more wired and/or wireless networks (e.g., a local area network (LAN), a wide area network (WAN), the Internet).

In some example embodiments, the computing system 400 can be used to execute various interactive computer software applications that can be used for organization, analysis and/or storage of data in various formats. Alternatively, the computing system 400 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 440. The user interface can be generated and presented to a user by the computing system 400 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A system, comprising:
at least one data processor; and
at least one memory storing instructions, which when executed by the at least one data processor, result in operations comprising:
determining, within a protein structure having a first sequence of residues, a first fixed segment and a first adjustable segment;
identifying a desired property associated with the protein structure;
generating, using a protein design computational model, a second sequence of residues comprising at least one of a corruption and a length change to the first adjustable segment; and
generating, using the protein design computational model, a modified protein structure having the second sequence of residues.

2. The system of claim 1, wherein the protein design computational model comprises a machine learning model trained, based on a plurality of known protein sequences, to approximate a data distribution populated by reduced dimension representations of protein sequences, and wherein the machine learning model generates the second sequence of residues by at least sampling the second sequence of residues from the data distribution.

3. (canceled)

4. The system of claim 2, wherein the sampling of the data distribution includes
generating a corrupted sequence by modifying the first adjustable segment but not the first fixed segment included in the first sequence of residues,
encoding the corrupted sequence to generate an encoding having a length corresponding to a quantity of residues present in the encoding,
generating an intermediate sequence by altering the length of the encoding of the corrupted sequence while maintaining a length of the first fixed segment, and
generating, based at least on a decoding of the intermediate sequence, the second sequence of residues, where the second sequence of residues is generated to include the first fixed segment.

5. (canceled)

6. (canceled)

7. The system of claim 4, wherein the operations further comprise:
decoding, based at least on an index map, the intermediate sequence, where the index map identifies the first fixed segment within the intermediate sequence, and wherein the decoding of the intermediate sequence includes determining, for each position within the intermediate sequence, a probability distribution across a vocabulary of possible amino acid residues.

8. (canceled)

9. (canceled)

10. The system of claim 2, wherein the operations further comprise:
determining, within the protein structure having the first sequence of residues, a second fixed segment; and
sampling the data distribution to generate the second sequence of residues to include the first fixed segment and the second fixed segment.

11. The system of claim 10, wherein the sampling of the data distribution includes
generating the corrupted sequence by modifying the first adjustable segment, where the corrupted sequence includes the modified first adjustable segment, the first fixed segment, and the second fixed segment;
generating the intermediate sequence by altering the length of the encoding of the corrupted sequence while maintaining the length of the first fixed segment or the second fixed segment;
generating an index map to identify the first fixed segment and the second fixed segment within the intermediate sequence; and
generating the second sequence of residues to include the first fixed segment and the second fixed segment by decoding the intermediate sequence based on the index map.

12. The system of claim 1, wherein a difference between a first length of the first sequence of residues and a second length of the second sequence of residues is distributed amongst the first adjustable segment and a second adjustable segment by at least changing a first length of the first adjustable segment and/or changing a second length of the second adjustable segment.

13. The system of claim 12, wherein the difference between the first length of the first sequence of residues and the second length of the second sequence of residues is determined based on a probability distribution of possible length differences between the first sequence of residues and the second sequence of residues.

14. The system of claim 12, wherein the difference between the first length of the first sequence of residues and the second length of the second sequence of residues is distributed proportionally or randomly across the first length of the first adjustable segment and the second length of the second adjustable segment.

15. (canceled)

16. The system of claim 12, wherein the difference between the first length of the first sequence of residues and the second length of the second sequence of residues is distributed to the first adjustable segment but not the second adjustable segment such that the second length of the second adjustable second segment is preserved.

17. The system claim 12, wherein the difference between the first length of the first sequence of residues and the second length of the second sequence of residues is distributed by applying no more than a maximum length change and/or no less than a minimum length change to at least one of the first length of the first adjustable segment and the second length of the second adjustable segment.

18. The system of claim 1, wherein the first sequence of residues comprises an antibody, and wherein the first segment comprises a complementarity determining region (CDR) of the antibody or a non-complementarity determining region of the antibody.

19. The system of claim 18, wherein an input of the protein design computational model includes one or more identifiers to enable a differentiation between residues comprising a first portion of the first sequence corresponding to a heavy chain of the antibody, a second portion of the first sequence corresponding to a light chain of the antibody, and/or a third portion of the first sequence corresponding to an antigen having a known binding affinity towards the antibody.

20. (canceled)

21. (canceled)

22. The system of claim 19, wherein the protein design computational model generates the second sequence of residues based on the one or more identifiers such that the first fixed segment included in the second sequence of residues is present in an identical chain as the first sequence of residues.

23. (canceled)

24. (canceled)

25. The system of claim 1, wherein the corruption includes at least one of inserting a residue into the first adjustable segment, deleting a residue from the first adjustable segment, and modifying a residue present in the first adjustable segment.

26. (canceled)

27. The system of claim 1, wherein the protein design computational model comprises an autoencoder.

28. (canceled)

29. The system of claim 1, wherein the operations further comprise:
identifying, based at least on the first fixed segment being associated with the desired property, the first fixed segment in the first sequence of residues.

30. (canceled)

31. The system of claim 1, wherein
the operations further comprise:
generating a fixed-length representation of the first sequence of residues including the first fixed segment and the first adjustable segment; and
applying the protein design computational model to generate the second sequence of residues by at least applying the at least one of the corruption and the length change to the first adjustable segment included in the fixed-length representation of the first sequence of residues.

32. The system of claim 31, wherein the fixed-length representation of the first sequence of residues is generated by at least
determining, based at least on a multi-sequence alignment including a plurality of known protein sequences, a global index having a plurality of integer positions,
assigning, based at least on the global index aligned to the first sequence of residues, a corresponding integer position from the plurality of integer positions to the each residue included in the first sequence of residues and
inserting, at each integer position where the first sequence of residues fails to include a corresponding residue, a gap character to indicate an absence of a residue at that integer position.

33. (canceled)

34. A computer-implemented method, comprising:
determining, within a protein structure having a first sequence of residues, a first fixed segment and a first adjustable segment;
identifying a desired property associated with the protein structure;
generating, using a protein design computational model, a second sequence of residues comprising at least one of a corruption and a length change to the first adjustable segment; and
generating, using the protein design computational model, a modified protein structure having the second sequence of residues.

35-66. (canceled)

67. A non-transitory computer readable medium storing instructions, which when executed by at least one data processor, result in operations comprising:
determining, within a protein structure having a first sequence of residues, a first fixed segment and a first adjustable segment;
identifying a desired property associated with the protein structure;
generating, using a protein design computational model, a second sequence of residues comprising at least one of a corruption and a length change to the first adjustable segment; and
generating, using the protein design computational model, a modified protein structure having the second sequence of residues.

68-110. (canceled)
